(19) United States
(12) Patent Application Publication
(10) Pub. No.: US 2025/0345485 A1
(43) Pub. Date: Nov. 13, 2025

(54) FIBROUS TISSUE DERIVED MATERIAL AND COMPOSITIONS COMPRISING SAME AND METHODS FOR MAKING AND USING SAME

(71) Applicant: Musculoskeletal Transplant Foundation, Edison, NJ (US)

(72) Inventors: Marc Gilles LONG, Monmouth JCT., NJ (US); Andrew Madans, Westwood, NJ (US)

(21) Appl. No.: 19/206,516

(22) Filed: May 13, 2025

Related U.S. Application Data
(60) Provisional application No. 63/646,263, filed on May 13, 2024.

(51) Int. Cl. A61L 27/36; A61L 27/58

(52) U.S. Cl. CPC A61L 27/3683; A61L 27/362; A61L 27/3633; A61L 27/58; A61L 2430/34; A61L 2430/40

(57) ABSTRACT

Methods of making fibrous tissue derived materials, as well as such materials and compositions comprising them, are provided. In addition to resizing, decellularizing, and delipidizing, tissue samples are subjected to one or more milling steps which are performed within certain parameters and using selected substances to control, retain, enhance, or a combination thereof, the fibrous quality of the resulting fibrous tissue derived material. Such fibrous tissue derived materials should provide beneficial handling properties, such as being a moldable putty when rehydrated, but having a fibrous texture, which is expected to be more cohesive than tissue derived materials subjected to more or longer milling steps, or those involving different substances during milling. The fibrous tissue derived materials and compositions comprising them provide a porous matrix or scaffold to support healing and remodeling of a subject's treatment, wound, defect, or reconstruction site.

### CROSS-REFERENCE TO RELATED APPLICATIONS

The present application claims the benefit of U.S. Provisional Application No. 63/646,263, filed May 13, 2024, the entire disclosure of which is incorporated by reference herein.

### FIELD OF THE INVENTION

The invention described and contemplated herein relates to fibrous tissue derived material and compositions comprising them, as well as methods for making and using the fibrous tissue derived material and compositions comprising them for treatment of wounds, including irregular and deep wounds and tissue defects, as well as for soft tissue reconstruction, plastic reconstruction, body contouring, and reconstruction.

### BACKGROUND

The use of graft materials often facilitates or enhances the effectiveness of treatment of various conditions involving damaged, diseased, or lost tissue. The goals of treatment typically include, but are not limited to, one or more of repair, replacement, augmentation, and reconstruction of tissue which has been damaged or lost due to disease, trauma, atrophy, surgery (e.g., excision of cancerous tissue, cosmetic surgery), and other causes. Some graft materials include naturally produced substances and materials, others include synthetic substances and materials, and still others include both. Some graft materials include one or more tissue derived materials which have been produced by processing one or more tissue samples recovered from one or more donors.

When administered to tissue at a treatment site, graft material may simply provide support or cover for tissue at a treatment site, while one or more biological processes, pathways, or responses proceed to repair, replace, or reconstruct damaged or lost tissue. On the other hand, tissue derived materials which form or are otherwise incorporated into graft material have also been found to promote, enhance, or otherwise influence biological processes, pathways, or responses at a treatment site. For example, tissue derived materials provide scaffolds with biological properties which support wound healing and soft tissue reconstruction, and also have growth factors, cells, and other substances which provide one or more of anti-inflammatory effects, host cell recruitment and attachment, and superior handling properties.

Of course, different conditions and the treatments applied to them will present and involve biological processes, responses, and pathways in different combinations and degrees of importance. Accordingly, different graft materials have been developed and designed having various characteristics and properties, in varying degrees and proportions, which are useful for affecting one or more of those biological processes, pathways, and responses, in selected ways. Some of the characteristics and properties of graft material useful for affecting biological processes, responses, and pathways, include being shapable, shape retention, cohesiveness, porosity, density, degradation rate, resorption rate, hydration rate, growth factor content, and many others.

Tissue derived materials, as well as processes for making such materials, are being developed to provide graft materials which are more fibrous and, consequently, provide a porous matrix with superior handling, structure, and biological properties to support or enhance wound healing or soft tissue reconstruction. Such properties include being formable into a mass or graft with increased cohesiveness and shape retention, even in the absence of crosslinking or other stabilizing treatments. Other properties include sufficient porosity and pore size for cell infiltration and incorporation when applied to a wound or tissue defect. Reduced degradation and/or absorption rates of the more fibrous tissue derived materials enable their use as grafts designed to have a longer period of residence time (i.e., retention time) at a treatment site during which beneficial effects of the fibrous tissue derived material on biological processes can be extended.

### BRIEF DESCRIPTION OF THE FIGURES

The present invention will be further explained with reference to the attached drawings, wherein like structures are referred to by like numerals and/or letters throughout the several views. The drawings shown are not necessarily to scale, with emphasis instead generally being placed upon illustrating the principles of the present invention.

FIG. 1 is an SEM image showing the fibrous structure of an exemplary embodiment of the fibrous tissue derived material produced from a human dermis tissue sample;

FIG. 2 is a flowchart showing the steps of an exemplary embodiment of a method for making fibrous tissue derived material, wherein milling steps are performed before and after decellularizing;

FIG. 3 is a flowchart showing the steps of a second exemplary embodiment of a method for making fibrous tissue derived material, wherein one or more milling steps are performed after decellularizing;

FIG. 4A is a flowchart showing the steps of one version of a third exemplary embodiment of a method for making fibrous tissue derived material, wherein one or more milling steps are performed after decellularizing and an alcohol is used during at least one milling step;

FIG. 4B is a flowchart showing the steps of another version of the third exemplary embodiment of a method for making fibrous tissue derived material, wherein one or more milling steps are performed after decellularizing and an alcohol is used during at least one milling step;

FIG. 5 is a flowchart showing the steps of a fourth exemplary embodiment of a method for making fibrous tissue derived material, wherein one or more milling steps are performed after decellularizing and dehydrating sheets of tissue, followed by one or more milling steps;

FIG. 6 is a photograph of cut up pieces of dehydrated dermis sheet such as are produced by the exemplary method of Example 5;

FIG. 7 is a photograph of dehydrated fibrous dermal tissue derived material produced from the cut up pieces shown in FIG. 6; and

FIG. 8 is a photograph of a quantity of the dehydrated fibrous dermal tissue derived material shown in FIG. 7 which has been rehydrated to form a shapable putty.

FIG. 9 is a photograph of the fibrous tissue derived material and a comparative prior art tissue derived matrix taken immediately prior to lyophilizing for visual comparison;

FIG. 10 is a photograph of the rehydrated fibrous tissue derived material which has been shaped and inserted into (and filling) a simulated tissue defect; and

FIGS. 11A and 11B are photographs of the fibrous tissue derived material, produced by the method of Example 6 and fully rehydrated with saline, being manually handled and manipulated.

### SUMMARY OF THE INVENTION

A fibrous tissue derived material is provided having a fibrous structure formed by extracellular matrix strands derived from processing one or more tissue samples, each of which comprises one or more soft tissues or portions thereof, wherein the fibrous structure comprises thin, wispy, flexible, elongated, and at least partially intertwined strands of the extracellular matrix, wherein the fibrous structure has a porosity greater than porosities of the one or more soft tissues of the one or more tissue samples, and wherein the porosity of the fibrous structure allows for fluid flow therethrough and facilitates migration and infiltration by fluids, bioactive substances, cells, and other beneficial materials, after administration of the fibrous tissue derived material to a treatment site. The fibrous tissue derived materials are at least partially decellularized.

The fibrous tissue derived material may be at least partially dehydrated, such as without limitation, lyophilized. In some embodiments, the fibrous tissue derived material is at least partially lyophilized and is capable of storage at temperatures above freezing for a period of time. The fibrous tissue derived materials may be combines with one or more biocompatible fluids.

The fibrous tissue derived material has one or more properties including: porous, compressible, cohesive, wickable, absorbent, moldable, shapable, cohesive, retains its shape, and combinations thereof. When the fibrous tissue derived material is at least partially hydrated or rehydrated, it has one or more properties including: porous, compressible, cohesive, wickable, absorbent, putty-like, moldable, shapable, cohesive, retains its shape, flowable, injectable, and combinations thereof.

In some embodiments, the soft tissues or portions thereof, which are used to produce the fibrous tissue derived material, may comprise one or more of dermis, adipose, fascia, muscle, and combinations thereof. The soft tissues or portions thereof, which are used to produce the fibrous tissue derived material, may consist essentially of dermis tissue. The soft tissues or portions thereof, which are used to produce the fibrous tissue derived material, may consist essentially of dermis tissue.

In some embodiments, the soft tissues or portions thereof, which are used to produce the fibrous tissue derived material, consist essentially of: about 1-99% dermis tissue and about 1-99% fascia tissue, not to exceed a total of 100% and based on the total weight of the tissue derived material. The soft tissues or portions thereof, which are used to produce the fibrous tissue derived material, consist essentially of: about 1-99% adipose tissue, about 1-99% fascia tissue, and about 1-99% dermis tissue, not to exceed a total of 100% and based on the total weight of the tissue derived material.

A method of treating a soft tissue condition is also provided and comprises administration of the fibrous tissue derived material or a composition comprising same to a treatment site of a subject, wherein the fibrous tissue derived material supports and enhances soft tissue healing, remodeling, and reconstruction.

Methods are provided for producing a tissue derived material having a fibrous structure and comprising extracellular matrix derived from processing one or more tissue samples, wherein the method comprises obtaining the one or more tissue samples, each of which comprises one or more soft tissues or portions thereof, each soft tissue having a native fibrous structure.

In some embodiments, the method further comprises the steps of: reducing the sample size of the one or more tissue samples one or more times, wherein at least one of the one or more times comprises performing one or more milling iterations using a milling device to produce the tissue derived material having a fibrous structure, wherein each of the one or more milling iterations is performed having milling parameters which retain, minimize or avoid destruction of, enhance, or a combination thereof, at least a portion of the native fibrous structure of one or more of the soft tissues in the one or more tissue samples; and at least partially decellularizing the one or more tissue samples by performing one or more decellularizing steps, sequentially, concurrently, or a combination thereof, wherein each of the one or more decellularizing steps comprises chemical decellularizing, physical decellularizing, or a combination thereof, and is different or the same as other decellularizing steps.

The one or more milling iterations may be performed before decellularizing, during decellularizing, before and during decellularizing, after decellularizing, during and after decellularizing, before and after decellularizing, or before, during, and after decellularizing.

The method further comprises increasing pH of the one or more soft tissue samples and thereby reducing acidity of the one or more tissue samples by either: combining one or more buffered aqueous solutions with the one or more tissue samples to produce a buffer-tissue mixture and subjecting the buffer-tissue mixture to at least one milling iteration, or performing at least one pre-mill soaking step by combining one or more buffered aqueous solutions with the one or more tissue samples to produce a buffer-tissue mixture and pausing a soaking period of time prior to subjecting the buffer-tissue mixture to at least one milling iteration, or performing at least one post-mill soaking step by combining one or more buffered aqueous solutions with the one or more tissue samples to produce a buffer-tissue mixture and pausing a soaking period of time prior to performing further processing steps.

In some embodiments of the method, each of the one or more milling iterations comprises either: milling parameters which include a milling period of time and a milling speed, and wherein the milling period of time is different from or the same as that of other milling iterations and the milling speed is different from or the same as that of other milling iterations, or two or more milling phases, each of which comprises milling parameters which include a milling period of time and a milling speed, and wherein the milling period of time is different from or the same as that of other milling phases and the milling speed is different from or the same as that of other milling phases.

In some embodiments of the method, at least one of the one or more milling iterations comprises: combining the one or more tissue samples with an aqueous solution selected from water, a buffered aqueous solution, or an alcohol solution, or a combination thereof, prior to operating the device at the milling parameters; and optionally removing at least a portion of the aqueous solution after completion of operating the device during each milling iteration, wherein the aqueous solution combined with the one or more tissue samples during a milling iteration is the different from or the same as the aqueous solution using in each of the other one or more milling iterations.

Some embodiments of the method further comprise forming a composition comprising the fibrous tissue derived material and having a three-dimensional shape which is simple, complex, or a combination thereof, wherein the composition has increased cohesiveness, in the substantial absence of crosslinking, as compared to a less fibrous tissue derived materials, and has porosity greater than porosities of the one or more soft tissues of the one or more tissue samples and which facilitates cell infiltration after administration to a treatment site.

### DETAILED DESCRIPTION

Fibrous tissue derived materials described and contemplated herein are produced from one or more human or animal tissue samples. Each of the one or more tissue samples processed to produce the fibrous tissue derived materials may comprise a single tissue type or multiple tissue types. Regardless of whether a tissue sample comprises a single type or multiple types of tissue, it may further comprise all or a portion of an initial tissue sample such that one or more selected or random portions of the initial tissue sample may be isolated to provide the one or more tissue samples which is/are then processed to produce the fibrous tissue derived material.

Suitable tissue types for the samples used for producing the fibrous tissue derived materials include soft tissues such as tissues which include or are formed by bundles of fibers, such as, without limitation, dermis, adipose, fascia, muscle, amnion, chorion, umbilical cord, placental disc and combinations thereof. It is noted that exemplary embodiments of the fibrous tissue derived materials, compositions comprising them, and methods for making and using them, are described in detail below starting with human dermis tissue samples. As already noted, it is contemplated that each tissue sample may include more than one type of tissue, such as a recovered tissue sample which includes epidermis, dermis, fascia, and adipose tissues, one or more of which may be separated and isolated for processing to produce the fibrous tissue derived materials. It should be further understood that it is well within the scope of the invention described and contemplated herein that other tissue types, such as but not limited to those mentioned above, are equally suitable for making and using the fibrous tissue derived materials.

The fibrous tissue derived materials and compositions comprising them provide porous matrices having improved handling, structure, and biological properties for supporting or enhancing wound healing, soft tissue reconstruction, or both. For example, the fibrous tissue derived material has one or more properties including, without limitation, porous, compressible, cohesive, wickable, absorbent, moldable, shapable, retains its shape, and when at least partially (or fully) hydrated, moldable, putty-like, shapable, cohesive, retains its shape, flowable, and injectable. More specifically, donor tissue samples are recovered from human or animal tissue sources and processed into a dehydrated fibrous tissue derived material which is capable of room temperature storage. Moreover, the fibrous tissue derived materials and compositions comprising them are biodegradable, bioresorbable, or both, upon application or implantation to a treatment site and during healing, remodeling, or both, of the host tissue at the treatment site. The fibrous tissue derived materials and compositions comprising them are capable of rehydration at the point of care (e.g., application or implantation) to provide a porous matrix or scaffold to support wound healing and soft tissue reconstruction.

In some embodiments, the fibrous tissue derived materials and compositions comprising them may be applied in an at least partially dehydrated form directly to the treatment site. On the other hand, they may be hydrated (i.e., not dehydrated or rehydrated after partial or full dehydration), in which form the materials and compositions have the properties of a flowable or putty-like form, prior to delivery to the treatment site. As will be described in further detail below, compositions including such fibrous tissue derived materials may further comprise one or more additional materials, depending on the particular properties and handling characteristics which would be beneficial base on the intended use of the compositions.

Methods of making the fibrous tissue derived materials, as well as compositions comprising them, generally include one or more resizing steps, one or more decellularizing steps, one or more disinfecting steps, one or more rinsing steps, and one or more milling steps. More particularly, to produce the fibrous tissue derived material, the milling steps are performed within certain parameters and using selected substances to control, retain, enhance, or a combination thereof, the fibrous quality of the tissue derived material produced by the method which will also be described in further detail below. More fibrous tissue derived materials are expected to provide beneficial handling properties, such as a moldable putty when rehydrated, but having a fibrous texture, which is expected to be more cohesive than tissue derived materials subjected to more or longer milling steps, or those involving different substances during milling, while still providing a porous matrix or scaffold to support healing and remodeling of the treatment wound, defect, or reconstruction site. The fibrous tissue derived material may be only partially dehydrated, or not dehydrated, or at least partially dehydrated and later further hydrated or rehydrated by combination with a biocompatible fluid, which provides an embodiment of the fibrous tissue derived material which provides one or more properties including, without limitation, moldable, flowable, injectable.

The fibrous tissue derived materials and compositions comprising them are different from tissue derived products which are intended to retain their original (i.e., final after recovery, cleaning, resizing, and other processing) or molded and packaged shape over time. Rather, the fibrous tissue derived materials and compositions comprising them has been developed and designed to become a porous matrix in a putty form upon hydration or rehydration. These matrices will resorb and remodel, after application or implantation, similar to natural dermis material which are often provided in sheet or patch forms. Without wishing to be limited by theory, it is believed that porosity and resorption rate of the fibrous tissue derived materials and compositions comprising them will depend on the shape and dimensions of the fibers, which can be tailored during the method of making them, as described hereinbelow.

The fibrous tissue derived materials and compositions comprising them are typically, but do not have to be, provided in dehydrated form and contained or packaged in any of several containers or devices. For example, without limitation, the fibrous tissue derived material and compositions comprising them may be packaged in a vial, a jar, a syringe, or an open barrel device with a plunger, or any other suitable container or storage or delivery device. Likewise, the fibrous tissue derived material and compositions comprising them may, for example without limitation, be delivered (e.g., applied or implanted) at a treatment site manually, using a device such as a spatula, or by syringe or an open barrel device with a plunger, or any other effective delivery device.

As used herein, the term “about” encompasses the explicitly recited amounts as well as deviations therefrom of ±10% of such explicitly recited amounts.

As used herein, the terms “absorbent” and absorbency” refer to the ability or capacity of a material to soak up and retain a liquid or substance such as but not limited to water, a solution, and other fluids.

The terms “administer,” “apply,” “implant,” “deliver,” and “place,” in all their grammatical forms, refer to placing, delivering, depositing, injecting, implanting, layering, spreading, coating, etc., a quantity of a substance or material on, in, adjacent to, or a combination thereof, a treatment site comprising a wound or otherwise damaged or injured tissue (i.e., host tissue) which is expected to benefit from such administration or implantation.

As used herein, an “aqueous solution” is a liquid containing water and, optionally, one or more additional solvents, substances, agents or materials. Accordingly, an aqueous solution may consist essentially of 100% water. Another non-limiting example is a solution comprising an alcohol mixed with some quantity of water, which is also a solvent and often simply referred to as an alcohol, but is also an aqueous solution for purposes of this disclosure. For example, without limitation, mixtures of 0.5% alcohol with 95.5% water (weight %), or of 25% alcohol with 75% water, or of 70% alcohol with 30% water, etc., where the alcohol is any one or more C₁-C₆ alcohols, are all aqueous solutions for purposes of this disclosure, as well as being solvents and alcohol solutions.

Generally and as used herein, the terms “biologically compatible” and “biocompatible” mean a material or substance which will not produce a toxic, injurious, or immunologic response when contacted with living tissue, such as when administered, placed, delivered, or implanted at a treatment site of a living subject (host). The terms “biologically compatible” or “biocompatible” are used herein interchangeably to describe any material or substance (liquid, solid, particulate, solution, gel, etc.) which does not cause an adverse or immunogenic reaction when contacted, administered, or implanted with host tissue. For example, biocompatible materials useful as hydration or rehydration fluids for combination with the fibrous tissue derived material and compositions comprising them include, without limitation, any diluent, carrier, etc., including without limitation a suitable solution, buffer, or excipient, preferably at point of care. Exemplary solutions include but are not limited to normal saline (0.9% sodium chloride), a physiological salt solution (phosphate buffered saline; PBS), Dulbecco's Modified Eagle Solution (DMEM), water, any autologous preparation (such as platelet rich plasma (PRP), bone marrow aspirate concentrate (BMAC), stromal vascular fraction (SVF)), corticosteroid, a solution containing hyaluronic acid (HA) or anti-inflammatory agents, and balanced salt solution (BSS).

The term “compressible” as used herein means that a material has the capacity to undergo a measurable reduction in thickness or volume when subjected to an external compressive force. This deformation may be partially or fully reversible. The compressive deformation may be Fully Reversible, with the material returning to near-original dimensions upon unloading; Partially Reversible, with some residual deformation; or Irreversible, where permanent changes occur due to the compressive force. This variability is influenced by factors such as crosslinking, hydration, duration or magnitude of applied force, or structural properties of the material. Compressibility can be characterized by Compression Strain (The percentage reduction in thickness under a defined compressive load, typically ranging from 5% to 40%), Compressive Modulus (the slope of the stress-strain curve in compression, typically expressed in kilopascals (kPa), with values for acellular dermal matrices may range from 10-300 kPa under physiological conditions), Bulk Modulus: (A measure of resistance to uniform compression, expressed in Pascals (Pa), and inferred from poroelastic behavior), or Load to compression (the force required to achieve a specific deformation, measured in Newtons (N), with typical values ranging from 20 N to over 100 N depending on graft thickness and orientation).

“Decellularizing” and “decellularized,” in all of their grammatical forms, as used herein, mean removing at least a portion of the endogenous cells and cellular material from a tissue sample. “Substantially decellularizing” and “substantially decellularized,” in all of their grammatical forms, mean that greater than about 50%, by weight (wt %), of the cellular DNA material endogenously present in a tissue sample is being removed, or has been removed, respectively, from the tissue sample, wherein wt % is based on the total weight of the cellular DNA material initially present in the recovered tissue before processing.

The terms “dehydrating” and “dehydrated” refer respectively to removal of at least a portion (i.e., a portion or substantially all) of water present in tissue, material, and compositions comprising one or both, and the condition of at least a portion of water present having been removed therefrom. A fully dehydrated fibrous tissue derived material has had substantially all of the water removed, so that the fully decellularized fibrous tissue derived material contains less than about 0.5% by weight of water (i.e., based on the total weight of the fibrous tissue derived material). Dehydrating may be performed by any of several techniques including, but not limited to, heating, air drying, desiccation, lyophilizing, and combinations.

The terms “delipidizing” and “delipidized,” as used herein in all of their grammatical forms, are any processes by which at least a portion of the lipids naturally present in a tissue are removed from the tissue, and describes a tissue sample from which at least a portion of lipids have been removed.

The term “derived” is used herein to describe circumstances in which a material or substance has been made from an original or intermediate material, tissue, or substance, for example, without limitation, through physical processing, chemical processing, or a combination thereof. The aforesaid processing may involve one, two, or even several steps or phases, as well as repeated and alternating steps or phases. For example, as described below, fibrous dermal tissue derived material is derived from one or more dermis tissue samples which may be subjected to one or more processing steps such as separation from other tissue types (e.g., from adipose, fascia, etc.), or even separation of a particular type or types of dermis (e.g., epithelial, reticular, etc.) from one or more dermis tissue samples, size reduction, decellularization, rinsing, disinfection, more rinsing, dehydration, and mixing with other materials, substances, components and structures. For example, without limitation, the one or more dermis tissue samples may be processed to remove epidermis layer(s) to produce full dermis layer tissue samples (i.e., without epidermis layer(s)), prior to further processing.

The term “diluent” refers to chemical compounds that are used to dilute the compound or composition of interest prior to delivery. Salts dissolved in buffered solutions (which also can provide pH control or maintenance) are utilized as diluents in the art, including, but not limited to a phosphate buffered saline solution and sodium chloride solutions.

The term “fibrous tissue derived material” is used herein to mean a material derived from one or more donor tissue samples which have been manipulated and processed to perform one or more steps of: recovering, separating, resizing, cleaning, delipidizing, decellularizing, disinfecting, reshaping, combining with one or more carriers, diluents, or other biocompatible materials which may be bioactive or not, where fibrous tissue derived material is comprised of thin, wispy, flexible, elongated, and at least partially intertwined strands of extracellular matrix. Fibrous tissue derived material may or not form or be formed into a porous body or mass of material.

The term “flowable” as used herein means a composition that is capable of being administered and reshaped or spread manually or using a spreading, coating, or injection device, without requiring significant mechanical force or structural modification. Flowable composition can be characterized by viscosity of (in the range of 0.1-100 Pa-s), yield stress (<500 Pa to allow deformation).

The terms “hydrate” and “rehydrate,” in all their grammatical forms, mean to add a biologically compatible liquid or gel, e.g., a diluent or carrier, to a material to provide a more malleable or flowable mixture comprising the material which has handling characteristics enabling easier administration or application of the mixture, whether manually, using an instrument such as a spatula, or passing the mixture through a cannula, syringe, or needle (i.e., injecting). Insufficient hydration or rehydration occurs when not enough liquid or gel has been added to a material for the resulting mixture to be administered or applied by the preferred method (e.g., passing through a cannula or injection through a syringe or needle, etc.). Overhydration occurs when the quantity of liquid or gel added to a material forms a mixture that lacks sufficient cohesiveness for effective and controlled administration or application by the preferred method (e.g., manual shaping or reshaping and placement, deposition and spreading using a spatula or other instrument, passing and controlled deposition through a cannula or syringe, etc.).

The term “injectable” as used herein means a composition that is capable of being administered through a syringe or needle, including via manual or mechanical force, without clogging or requiring modification of the composition's structure or temperature. Injectable compositions can be passed through a syringe or needle having gauge sizes of 16-27 gauge, by application of reasonable force (manually or otherwise), i.e., without requiring excessive force, such as greater than about 50 Newtons, at room temperature.

The terms “lyophilizing” and “lyophilized” refer to the process of freeze drying which includes a freezing phase and one or more drying phases, and the condition of having been subjected to a lyophilizing process. Lyophilizing often enables or prolongs the preservation of a tissue, material, or substance, for a period of time longer than without lyophilizing and with storage at temperatures above freezing (e.g., above 0° C.).

The terms “milling” and “blending,” unless otherwise indicated, are used herein interchangeably to mean a size reduction step which generally produces smaller sized tissue pieces than simple cutting or slicing with a scalpel or knife, and is performed using any device which performs blending or milling using knifes, blades, linear, arcuate, or circular (e.g., rotating) cutting edges, or some combination thereof, and is less likely to damage the native fibrous structure of the initial tissue sample(s) than, for example, grinding techniques and devices (balls, plates, etc.).

The term “pharmaceutically acceptable,” as used herein, refers to a material which is relatively nontoxic, i.e., the material may be administered to an individual without causing undue undesirable biological effects or interacting in a deleterious manner with any of the components of the composition in which it is contained.

The term “point of care” is used herein to mean at or near the point in time when a clinician or other health care provider administers health care services and/or products, including the composition of the invention, to a patient.

As used herein, the terms “room temperature” and “ambient temperature” are used interchangeably to mean any one or more temperatures above freezing and above typical refrigeration temperatures (e.g., from about greater than 0° C. to about 10° C.), for example from greater than 0° C. to about 40° C., and more typically, but not limited to, from greater than about 18° C. to about 30° C.

“Sterilization” and “sterilizing” as used herein, in all of their grammatical forms, is any process that renders an object (e.g., a tissue, a container for tissue, or an implement for processing tissue) essentially free from pathogenic organisms and/or viruses by destroying them or otherwise inhibiting their growth or vital activity. Such processes may include exposure of the object to one or more, without limitation, of gamma radiation, electron beam radiation, x-ray radiation, chemical agents (e.g., alcohol, phenol, ethylene oxide gas, acids, bases, or peroxides), heat, or ultraviolet radiation for sufficient duration and dosages. When sterilization is performed on a finished tissue product in its final packaging, the process may be referred to as “terminal sterilization”.

As used herein, the term “storing,” whether used for tissue samples recovered from a donor (i.e., before or after any processing steps, including contacting with one or more protectants), or preserved tissue samples or preserved tissue forms comprising same (i.e., after lyopreserving), includes any periods of transport or shipping, regardless of temperature.

The term “therapeutically acceptable” with respect to a formulation, composition or component, as used herein, means having no persistent detrimental effect on the general health of the subject being treated.

The term “therapeutically effective amount,” as used herein, refers to a sufficient amount of an agent or a compound or composition being administered which will relieve, partially or fully, one or more of the symptoms of the disease or condition being treated, e.g., tissue damage or associated pain or other symptoms or causes of the treated disease.

The terms “wickable” and “wickability” as used herein mean a material or composition capable of spontaneously transporting liquid through its structure by capillary action, without the application of external pressure or suction.

In the case of conflict, the present specification, including definitions shall control. In addition, the particular embodiments discussed below are illustrative only and not intended to be limiting.

The articles “a” and “the” are not to be construed as limiting the subject feature to a single element, step, item, etc.

When describing the performance of a method step “before” or “after” another step or steps, such description should not be construed to mean immediately before or after another step or steps. In other words, when one method step is described as being performed “before” another step, one or more other method steps may be performed in between the aforesaid one step and another step. Analogously, when one method step is described as being performed “before” another step, one or more other method steps may be performed in between the aforesaid one step and another step. When one method step is described as being performed “before” another step, one or more other method steps may be performed in between the aforesaid one step and another step. When one method step is described as being performed “after” another step, one or more other method steps may be performed in between the aforesaid another step and one step.

The fibrous tissue derived materials described and contemplated herein are produced from human or animal tissue samples. As already mentioned, suitable tissue types for the samples used for producing the fibrous tissue derived materials are generally soft tissues which include or are formed by bundles of fibers, and include, without limitation, dermis, adipose, fascia, muscle, and combinations thereof.

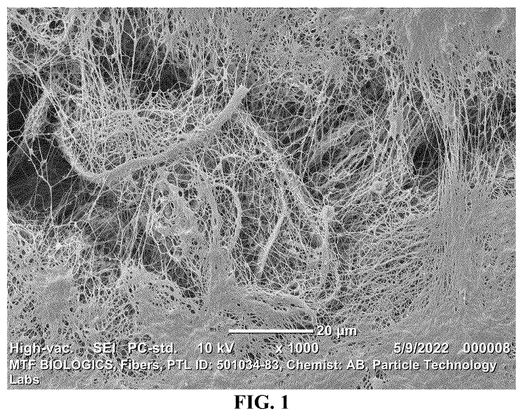

The fibrous structure of the fibrous tissue derived material described and contemplated herein is formed of thin, wispy, flexible, elongated, and at least partially intertwined strands of extracellular matrix, as shown in the SEM image provided in FIG. 1. The material shown in FIG. 1 is a fibrous tissue derived material made from a dermis tissue sample, according to a method (described in detail hereinbelow) which included size reduction, decellularizing, disinfection, milling and dehydration. This fibrous structure is different from material comprising a plurality of fibers which are generally separate and discrete elongated elements or fibers, such as some bone tissue derived fiber materials or some cartilage derived fiber materials (some of which are produced by grating or slicing to produce a plurality of substantially individual, discrete elongated pieces of tissue which may be described as fibers, strings, ribbons, etc.).

The structure of fibrous tissue derived material is likely to be altered or damaged by crosslinking and additional dehydrating steps, from a fibrous structure such as that shown in FIG. 1, to a more ribbon-like structure with less cohesiveness upon rehydration. Accordingly, minimal, if any, crosslinking and hydration are preferred to produce the fibrous tissue derived material described and contemplated herein.

The fibrous tissue derived materials and compositions including them provide porous matrices having superior handling, structure, and biological properties to support or enhance wound healing or soft tissue reconstruction. In one exemplary embodiment, donor tissue samples are recovered from human or animal tissue sources and processed into a dehydrated (e.g., lyophilized) fibrous form which are capable of room temperature storage, and have cohesive and porous properties. When rehydrated, the dehydrated fibrous tissue derived material and compositions comprising them are cohesive and putty-like, as well as being porous which, after delivery or implantation at a treatment site, allows for fluid flow therethrough and migration and infiltration by fluids, bioactive substances, cells, and other beneficial materials characteristics, which supports and enhances wound healing and soft tissue remodeling and reconstruction.

Alternatively, the fibrous tissue derived material may be produced by another exemplary method which does not include dehydrating, or which includes only partially dehydrating, the tissue being processed. This produces fibrous tissue derived material which is already at least partially hydrated. Such hydrated or partially hydrated fibrous tissue derived materials may still be further hydrated by contact or combination with one or more biocompatible fluids prior to or at the point of delivery to the treatment site.

The fibrous tissue derived material is substantially decellularized, which means that a majority of the endogenous cells and cellular material (i.e., greater than 50 wt % of the originally present cellular DNA material) have been removed from the sample tissue during processing, Accordingly, in addition to providing a resorbable three dimensional scaffold for cell infiltration and new tissue ingrowth, the fibrous tissue derived material lacks immunogenicity and is, therefore, highly biocompatible. In some embodiments, without limitation, greater than about 80 wt %, or greater than about 90 wt %, or greater than about 95 wt %, of the originally present cellular DNA material has been removed from the fibrous tissue derived material, which means the material contains less than about 20 wt %, or less than about 10 wt %, or less than about 5 wt %, of its originally present cellular DNA material.

The fibrous tissue derived material may also be disinfected, for example, by contacting the tissue sample with a disinfecting solution comprising one or more disinfecting agents, such as without limitation, antibiotics, alcohols, a peroxy compound (e.g., peracetic acid, PAA), and a detergent or surfactant. PAA is an effective selection for disinfecting tissue samples without damaging their native (natural) fibrous structure,

The fibrous tissue derived materials are comprised of thin, wispy, flexible, elongated, and at least partially intertwined strands of extracellular matrix, which form a porous body, mass, or bundle of the fibrous tissue derived material. This porous body may be formed into any simple or complex three-dimensional shape or structure, including but not limited to one or more of: a symmetrical shape, an asymmetrical shape, an irregular shape, a sheet, a layer, a film, an irregular mass, a block, a disk, a puck, a cylinder, a cube, a cone, a dome, a cuboid, a sphere, a shape of an anatomical structure, a portion of any of the foregoing shapes, and a combination thereof. In some embodiments, the final three-dimensional shape of a composition comprising the fibrous tissue derived material is monolithic, or partially monolithic. On the other hand, the composition may have a final three-dimensional shape composed of a plurality of shapes, such as but not limited to those previously mentioned. The fibrous nature of the fibrous tissue derived materials and compositions comprising them is expected to provide increased cohesiveness and shape retention, without crosslinking, as compared to a less fibrous tissue derived materials, while also providing porosity for cell infiltration.

Due to their differing compositions (e.g., different types and proportions of proteins such as, but not limited to, collagen Types I-VI, glycosaminoglycans, proteoglycans, growth factors, elastin, fibronectin, proteoglycans, etc.) different soft tissues may provide or facilitate different (but sometimes overlapping) properties and biological activities when processed to provide the fibrous tissue derived materials described and contemplated herein. Without wishing to be limited by theory, dermis derived matrices (such as fibrous dermis derived material) may provide or facilitate formation of new tissue matrix (e.g., probably through facilitating or promoting endothelial cell infiltration), which may, in turn, facilitate or promote angiogenic activity after implanting or other administration. Adipose derived matrices (such as fibrous adipose derived material) may provide or facilitate adipogenic activity after implanting or other administration, and fascia derived matrices (such as fibrous fascia derived material) may provide or facilitate angiogenic activity and also may provide greater volume retention at a treatment site after implanting or other administration. Also without wishing to be limited by theory, both adipose and fascia tissues facilitate angiogenic activity, but fascia does so to a greater degree than adipose, which means that selection and inclusion of both adipose and fascia tissue types will increase angiogenic potential of the resulting fibrous tissue derived material. The aforementioned properties and biological activities provided by dermis, adipose, and fascia tissues are not limited to those stated and, furthermore, may also be provided by other types of soft tissue. Additionally, other types of soft tissue may provide other properties and biological activities instead of or in addition to, the aforementioned properties and biological activities.

It is within the ability of persons of ordinary skill in the relevant art, based on the general knowledge in the relevant art and this disclosure, to select which soft tissues should be obtained as tissue samples for processing as disclosed and contemplated herein to produce the fibrous tissue derived materials having desired properties and facilitating biological activities which are selected based on the intended uses (i.e., types of treatment, type of condition being treated, or both) of the fibrous tissue derived materials and compositions comprising them.

As disclosed above, it is contemplated that the one or more starting tissue samples from which fibrous tissue derived materials are produced may comprise more than one type of soft tissue. Further, depending on the intended use, predetermined proportions (ratios) of two or more types of tissue may be varied and selected, also based on one or more of the desired properties, desired biological activities, or both provided by different types of soft tissue, and the intended uses of the fibrous tissue derived material. Determining suitable proportions of the two or more types of soft tissue selected for processing to produce the fibrous tissue derived material will depend on balancing and prioritizing the desired properties, benefits, and biological activities provided by each of the selected types of soft tissue.

For example, without limitation, in an exemplary embodiment, the one or more tissue samples may include dermis and fascia for processing to produce fibrous tissue derived materials capable of facilitating both new tissue matrix formation and angiogenic activity due to the dermis, as well as increased volume retention due to the fascia, upon implanting or other administration of the fibrous tissue derived material at (or proximate) a treatment site. Furthermore, the dermis tissue and fascia tissue may be combined, each in a predetermined proportion of about 1-99%, not to exceed a total of 100% and based on the total weight of the tissue derived material, depending on the intended use and whether one or the other of angiogenic activity and increased volume retention is of greater interest or importance. Implantation of fibrous tissue derived material derived from fascia tissue, without any dermis combined therewith (i.e., 100 wt % fascia tissue samples) will maximize volume retention. Of course, as will be understood by persons of ordinary skill in the relevant art, the benefits and properties provided by the fascia tissue derived component of such fibrous tissue derived materials (i.e., adipogenesis, increased volume retention) will be lessened as the proportion of fascia derived material in the overall fibrous tissue derived material is decreased.

For example, without limitation and without being limited by theory, in an exemplary embodiment, three soft tissue types may be selected such as adipose, fascia, and dermis tissues to produce fibrous tissue derived materials capable of facilitating adipogenic activity, increased tissue matrix formation and volume retention, and angiogenic activity, respectively, upon implanting or other administration of the fibrous tissue derived material at (or proximate) a treatment site. Furthermore, the starting tissue samples and resulting fibrous tissue derived material may include about 1-99% adipose tissue, 1-99% fascia tissue, and 1-99% dermis tissue, not to exceed a total of 100% and based on the total weight of the tissue derived material. For example, without limitation, where adiopgenic activity is prioritized, and angiogenic activity and increased volume retention are of comparable importance, the adipose tissue may be present in a proportion of about 75-99%, while each of the fascia tissue and dermis tissue may, independently of one another, be present in a proportion of above 1-24%, not to exceed a total of 100% and based on the total weight of the tissue derived material.

Furthermore, compositions comprising the fibrous tissue derived material may have a final selected three-dimensional shape, but may be produced in multiple components, each having their own three-dimensional shape and which, when assembled together, will form the composition having the final selected three-dimensional shape. In such multiple component embodiments, the disassembled shaped components may be packaged independently of one another but collected, stored and shipped together in a kit. This may facilitate some or all of processing, storage, delivery, and use, of the compositions comprising fibrous tissue derived material. For example, without limitation, the final composition may be a collection of small cylinders packaged together to form a final structure of constituent components. Even when including multiple components or pieces, the final composition should still provide a cohesive support structure or scaffold when all pieces are prepared, assembled, mixed or otherwise combined, and rehydrated. Such embodiments permit inclusion of one or more components comprising the same or different tissue derived materials, whether fibrous or not, and even comprising one or more other biocompatible materials and substances, for combination to produce compositions comprising fibrous tissue derived material, as described and contemplated herein.

When placed or implanted at a treatment site comprising a wound or tissue defect to be treated, the fibrous tissue derived materials and compositions comprising them will provide a scaffold that will be resorbed and remodeled during tissue healing and remodeling. Moreover, the fibrous tissue derived materials and compositions comprising them will be especially suitable and advantageous for use to treat deeper or irregularly shaped wounds, due to its intentional flowability and formability, as well as tissue defects requiring bulk tissue to fill voids.

The fibrous structures which form the fibrous tissue derived materials may be assessed, such as by SEM imaging during tissue processing and production to qualitatively assess the fibrous quality of the final material as compared to less fibrous formulations (e.g., longer, fuller fibrous structures, and/or greater intermingling of these fibrous structures). It is expected that the more fibrous and cohesive tissue derived material will hold its shape and bulk together (have shape retention), even without crosslinking, better than a less fibrous formulation. The degree of cohesiveness and shape retention of the fibrous tissue derived materials and compositions comprising them and their ability to maintain their shape, at least until intentionally reshaped, can be explored and measured by studying and measuring the following properties:
Enzyme degradation: the more fibrous and cohesive the fibrous tissue derived material is, the longer it is expected to take to degrade (i.e., a slower degradation rate is expected), after placement or implanting at a treatment site at which tissue healing and remodeling is expected and desired. Measurement of such a property would, for example, be conducted by placing mass normalized samples of the material in physiologically relevant enzyme solution(s) and assessing the length of time required to fully dissolve or dissociate for each sample and/or quantification of the amount of mass remaining from each sample after a specified time period.
Cohesiveness: a more fibrous and cohesive tissue derived material should retain more of its bulk during a physical smearing test, as compared to a less fibrous formulation. The general procedure of a physical smearing test to demonstrate the aforesaid correlation between increased fibrousness and increased cohesiveness would be to spread samples of fibrous tissue derived samples having varied fibrousness across a given surface using a standardized force/technique and determine how far each sample spreads and/or how much of the material remains in the original mass (or body) after spreading.
Mechanical testing: a more fibrous and cohesive fibrous tissue derived material is expected to have greater tensile strength and greater compressive strength than a less fibrous material. This property correlation would be demonstrated via well known mechanical testing procedures, using an instrument such as an Instron testing machine.

In some embodiments of the fibrous tissue derived material, such as those intended for use in treatments where facilitated or enhanced cell migration and infiltration would be particularly beneficial, the fibrous tissue derived material may have sufficient porosity and pore size for cell infiltration and incorporation when applied to a wound or tissue defect (e.g., >80% porosity, and about 75-400 um median pore size). These features can be assessed and verified using SEM imaging, mercury intrusion, Micro-CT imaging, and other well-known characterization methods.

Compositions including the fibrous tissue derived materials described and contemplated herein may further comprise one or more additional materials, depending on the particular properties and handling characteristics which would be beneficial based on the intended use of the compositions. Such additional materials should be biocompatible and include, without limitation, biocompatible non-tissue materials, including but not limited to polymers (natural or synthetic), ceramics, metals, nature-derived or animal-derived biomaterials. The compositions including the fibrous tissue derived materials may also contain endogenous beneficial substances such as growth factors, extracellular matrix components, nutrients, biologically active molecules, vitamins, or integrins which facilitate various tissue healing and remodeling mechanisms including, without limitation, extracellular matrix production and deposition, cell infiltration and proliferation, pathogen barrier and reduction, and angiogenesis.

As previously described, one or more fibrous tissue derived materials, may be contacted, added, mixed, layered, coated, or otherwise combined, with one or more additional materials, either during production of the composition, or by packaging and collecting them into a multiple component kit for such combining at the point of use or delivery to a treatment site.

Furthermore, the compositions including the fibrous tissue derived materials described and contemplated herein may be coated with, infused with, or otherwise include exogenous substances or materials, including without limitation, cells, growth factors, extracellular matrix components, nutrients, integrins, anti-microbial agents, anti-infective agents, bacteriostatic agents, or other substances such as, but not limited to, those which promote cell migration, attachment, proliferation, growth and activity. For example, without limitation, some growth factors are known and/or believed to expedite cell recruitment, modulate inflammation, etc. Methods for making the tissue derived porous matrices and using them for wound treatment are also described herein below.

Biocompatible materials useful as hydration or rehydration fluids for combination with the fibrous tissue derived materials and compositions comprising them, as described and contemplated herein, include, without limitation, any biocompatible diluent, carrier, solution, buffer, or excipient. Suitable hydration or rehydration fluids include, without limitation, one or more of: normal saline (0.9% sodium chloride), a physiological salt solution (phosphate buffered saline; PBS), Dulbecco's Modified Eagle Solution (DMEM), water, any autologous preparation (such as platelet rich plasma (PRP), bone marrow aspirate concentrate (BMAC), stromal vascular fraction (SVF)), hyaluronic acid (HA) solution, balanced salt solution (BSS), blood, and blood marrow. Furthermore, a hydration or rehydration fluid may include one or more other additional materials or substances, including but not limited to: antibiotic agents, antimicrobial agents, anti-inflammatory agents, anti-coagulant agents, cells, cell components, growth factors, and any number of other beneficial substances,

There are known methods for producing porous tissue derived matrices which are generally particulate, homogenous and readily dehydrated to provide shaped porous tissue derived matrices, including without limitation, the methods disclosed in WIPO Publication No. WO2020227601, which is the publication of International Patent Application No. PCT/US2020/032022, filed May 8, 2020. These porous tissue derived matrices are resorbable, have a plurality of interconnected pores which allow fluid flow through the matrix, and are produced by methods which generally comprise the steps of: reducing the size of a tissue sample; decellularizing the tissue sample; further reducing the size of the decellularized tissue, forming or modifying pores in the tissue to produce a porous matrix, and stabilizing the porous matrix. Further reducing the size of a tissue sample may be performed by blending, milling, or both. The porous matrix may be stabilized by dehydrating, crosslinking and again dehydrating, or both. In some embodiments, dehydrating and forming or modifying pores may be performed concurrently by lyophilizing decellularized tissue. In some embodiments, the aforesaid methods also comprise the step of disinfecting the tissue.

It has been found that making one or more of several modifications to the tissue processing methods described in WO2020227601 developed methods for producing fibrous tissue derived material, which retains at least a portion of the natural fibrous structure of the starting tissue samples, while still being resorbable and porous. These fibrous tissue derived materials are cohesive, i.e., have the ability to maintain volume and shape even without dehydration or crosslinking, and porous, i.e., promote cell migration and cell infiltration from host tissue after administering or implanting the material at a treatment site.

Generally, as compared to the methods disclosed in WO2020227601, the presently described and contemplated methods include one or more of the following modifications:
(1) raising pH (i.e., reducing the acidity) of the tissue sample during one or more steps of blending/milling the tissue by;
   (a) adding one or more steps of blending/milling the tissue where the tissue is in a buffered solution (rather than just water), and
   (b) replacing water with a buffered solution in one or more existing steps of blending/milling the tissue;
   (c) adding a soaking step, with the tissue in a buffered solution for a soaking period of time, before or after one or more steps of blending/milling the tissue;
(2) reducing the time period for one or more existing steps of blending/milling the tissue;
(3) reducing the iterations (number of times performed) of one or more existing steps of blending/milling the tissue;
(4) reducing the speed (rpm) during at least a portion of one or more existing steps of blending/milling the tissue; and
(5) eliminating one or more existing steps of blending/milling the tissue.

It should be understood that, as will become clearer based on the description below, any method step of further reducing the size of the tissue which is performed by blending, milling, or a combination thereof (more concisely referred to hereinafter as “milling”), often (but does not have to) includes more than one stage or phase, each of which is defined by a different time period, a different speed, or both, as compared to other stages or phases performed in that blending or milling step. For example, a step of milling the tissue may comprise a first phase in which the tissue is milled for about 10-30 seconds, such as about 15-25 seconds, at about 1000-3000 rpm, such as about 1500-3000 rpm, followed by a second phase in which the tissue is further milled for about 45 seconds to about 5 minutes, such as about 1.5-2.5 minutes, at about 3000-5000 rpm, such as about 3500-4500 rpm. Furthermore, it should be understood that any one or more of the above-stated modifications to a milling step may be applied to one or more phases of the milling steps, or to the entire milling step.

Generally, the fibrous tissue derived material described and contemplated herein are made from one or more tissue samples by a method which generally includes the initial step of obtaining one or more tissue samples comprising a desired tissue type or portion thereof, reducing the size of the tissue samples, optionally delipidizing, chemical decellularization (e.g., using hypertonic solution, such as sodium chloride solution, one or more detergents, etc.), chemical disinfection (e.g., peracetic acid (PAA), ethylene oxide, etc.), and one or more milling steps (which may be referred to herein as “further reducing the size of the tissue”).

Each of the one or more milling steps may be performed before one or more of the other steps, after one or more of the other steps, or both. The milling step or steps are generally, performed with the tissue in a solvent or solution and using a cutting type of blending or milling device, followed by, optionally, concentrating the milled tissue (e.g., removing solvent or solution by centrifuging or otherwise separating the milled tissue from at least a portion of the solvent or solution), and finally lyophilizing or otherwise dehydrating the milled tissue to form the fibrous tissue derived material. While shaping the dehydrated milled tissue (e.g., in a mold or otherwise), followed by at least partially crosslinking and dehydrating the tissue again to maintain its shape may be performed, it is preferred that such processing steps are minimized or avoided since the fibrous characteristic of the dehydrated milled tissue may be diminished by such crosslinking and repeated dehydration. Furthermore, it is contemplated that the fibrous tissue derived material and compositions comprising it have sufficient shape retention ability to render crosslinking unnecessary or at least, less important.

Methods for making the fibrous tissue derived material and compositions comprising the fibrous tissue derived material, including several exemplary embodiments, will now be described.

It should be noted that, unless otherwise stated or indicated, the order in which method steps are listed and their labels (e.g., (A), (B), . . . and (1), (2), . . . ) do not indicate or require that the method steps be performed in any particular order. Rather, unless otherwise stated, any one or more of the method steps may be performed in any order, relative to the others, and may be performed more than once. For example, without limitation, the step of (B) reducing the size of the tissue sample may be performed one or more times between any of the other method steps, as well as before and after any of the other method steps. Furthermore, it is possible and contemplated that one or more method steps may be performed concurrently, such as, without limitation, performing the steps of reducing the size of the tissue sample and decellularizing the tissue sample concurrently.

Methods for making a fibrous tissue derived material which is cohesive, porous, resorbable, and forms a shapable putty when rehydrated, most generally comprise the steps of:
(A) obtaining a tissue sample;
(B) optionally, reducing the size of the tissue sample;
(C) optionally, delipidizing the tissue sample;
(D) milling the tissue sample at least once, with or without contacting the tissue sample with a buffered solution, and with or without first performing a pre-mill soaking step in an aqueous solution;
(E) optionally, removing solution or solvent from the tissue sample;
(F) decellularizing the tissue sample;
(G) optionally, disinfecting the tissue sample;
(H) optionally, combining an aqueous solution or solvent with the tissue sample;
(I) optionally, removing solution or solvent from the tissue sample; and
(J) optionally, dehydrating the tissue sample to form the fibrous tissue derived material.

The step of (A) obtaining a tissue sample comprising a desired tissue type comprises obtaining one or more tissue samples, each of which comprises one or more desired tissue types. Tissue types suitable for the one or more tissue samples are soft tissues which include or are formed by bundles of fibers, such as, without limitation, dermis, adipose, fascia, muscle, and combinations thereof. In some embodiments, for example without limitation, one or more tissue samples may be obtained, each of which comprises dermis tissue. In some embodiments, one or more tissue samples may be obtained, each of which comprises both adipose tissue and fascia tissue. In still other embodiments, two or more tissue samples may be obtained, at least one of which comprises dermis tissue, and at least one of which comprises fascia tissue.

Furthermore, it should be understood that the step of (A) obtaining a tissue sample comprising a desired tissue type may involve performing one or more of the following preliminary processing steps: recovering one or more tissue samples from one or more human or animal donors, cleaning the one or more tissue samples to remove undesirable substances (e.g., blood clots, blood components, debris, storage solution, etc.), separating and removing undesired tissue types from the one or more tissue samples, and separating and retaining one or more desired portions, sections, or layers, of the one or more tissue samples, to produce the one or more tissue samples comprising one or more desired tissue types.

In some embodiments, one or more of the foresaid preliminary processing steps may have already been performed so that it is necessary only to perform one or more remaining preliminary processing steps, to obtain the one or more tissue samples ready to be subject to further steps of the method described and contemplated herein. In some embodiments, all of the foresaid preliminary processing steps have already been performed so that the one or more tissue samples are readily available and can be procured in ready-to-process condition in performance of the step of (A) obtaining the one or more tissue samples.

The step of (B) reducing the size of the tissue sample may be performed for any number of reasons including, but not limited to, facilitate handling and further processing, provide a suitable or desired physical form in preparation for a subsequent step or to facilitate a subsequent step being more effective, providing a physical form suitable or desired for the final fibrous tissue derived material, and combinations thereof. Modification and reduction of the size of the tissue sample may be accomplished by any suitable and effective techniques. For example, without limitation, it is often useful to (B) reduce the size of the tissue sample early in the method, such as without limitation, by cutting the tissue with a scalpel or similar instrument, into smaller strips or pieces to facilitate further treatment and processing steps. Blending and milling are also possible during the step (B) of reducing the size of the tissue sample, though maybe at low speed or of short duration. The tissue samples may be frozen prior to cutting the samples into smaller strips or pieces.

Performing the size reduction step (B) typically, but is not required to, produce smaller tissue pieces having dimensions (e.g., length, width, diameter, etc.) on the order of centimeters (cm), such as without limitation from about 0.5 cm to about 30 cm, including any one or more ranges or values therebetween and including the stated endpoints. The tissue pieces produced by a size reduction step (B) may have dimensions, for example without limitation, of about 0.5-20 cm, or about 0.5-10 cm, or about 0.5-5 cm, or about 1-10 cm, or about 1-5 cm, or about 0.5-4 cm, or about 1-4 cm, or about 0.5-3 cm.

Additionally, or alternatively, the size reduction step (B) may be performed to isolate one or more desired portions or sections of one or more tissue samples, prior to further processing. For example, without limitation, a full thickness dermis tissue (i.e., including at least epidermal and dermal layers, and possibly hypodermis and/or fascia) may be subjected to physical, chemical, or both, techniques which isolate the dermis layers, such as by removing the epidermis and epithelial layers, as well as the hypodermis and fascia, if present. In some embodiments, physical, chemical, or both, techniques may be performed to isolate a particular sublayer of the dermis, for example without limitation, the reticular dermis layer, or the papillary dermis layer, from the other layers of a full thickness dermal tissue sample.

For embodiments which produce fibrous tissue derived material which includes more than one type of soft tissue or portions thereof, the one or more tissue samples to be processed according to the method disclosed and contemplated herein may have any of several compositions and combinations of the desired soft tissues. In some embodiments, without limitation, one or more tissue sample(s) may be obtained wherein each comprises one or more of the desired selected soft tissue types or portions thereof so that collectively, the one or more tissue samples comprise the desired types and proportions of selected soft tissue types desired in the fibrous tissue derived material. In some embodiments, several tissue samples may be obtained wherein at least a first one of them comprises a first selected soft tissue type or portion thereof, at least a second one of them comprises a second selected soft tissue type or portion thereof, and so on, so that collectively, the several tissue samples comprise the desired types and proportions of selected soft tissue types desired in the fibrous tissue derived material.

Furthermore, any of the tissue samples may generally be subjected to further processing steps (e.g., milling, decellularizing, delipidizing, rinsing, disinfecting, etc.) together or separately. For example, without limitation, adipose and fascia tissue samples may be processed together through one or more of the further processing steps, or separately through selected steps such as if it were desired to more fully delipidize the adipose samples before combining with the fascia samples for additional processing steps. Similarly, without limitation, adipose and dermis samples may be processed together through one or more of the further processing steps, or separately through selected steps such if it were desired to more fully mill the dermis samples, prior to combining the adipose samples for additional processing steps.

Furthermore, where three types of soft tissue samples have been selected and obtained, samples of all three soft tissue types may be processed together through one or more of the processing steps, or any two may be processed together through one or more of the processing steps before adding samples of the third soft tissue type for additional processing steps. Of course, samples of each selected soft tissue type may be subjected to one or more processing steps separately from the others before being combined, for example, to be mixed or blended together, or to be subjected to final one or more final milling iterations or one or more rinsing steps, and any of many other possible arrangements as will be recognized and determinable by persons of ordinary skill in the relevant art.

Size reduction steps (B) may also be performed multiple times such as, for example without limitation, producing tissue pieces having lengths and widths, independently of one another, from 5 to 20 cm, including any one or more ranges or values therebetween and including the stated endpoints, followed by further size reduction to produce tissue pieces having lengths and widths, independently of one another, from 0.5 to 10 cm, including any one or more ranges or values therebetween and including the stated endpoints.

The method for producing the fibrous tissue derived material may further include the step of (C) delipidizing the tissue sample, using techniques known now or in the future to persons of ordinary skill in the relevant art. Delipidizing decreases the amount, or removes substantially all, of the lipids initially present in the tissue sample. A fully delipidized fibrous tissue derived material has had substantially all of the lipids native to the one or more starting tissue samples removed, so that the fully delipidized fibrous tissue derived material contains less than about 0.5% by weight of native lipids (i.e., based on the total weight of the fibrous tissue derived material). Whether delipidizing is performed will generally be determined according to the type of tissue being treated (e.g., adipose, dermis sample including hypodermis, etc.) and the intended final use of the tissue derived porous matrix. In some embodiments, but not necessarily, the step of (C) delipidizing the tissue sample is performed prior to the step of (F) decellularizing the tissue sample. In some embodiments, the step of (C) delipidizing is not performed.

The step of (C) delipidizing the tissue sample may, for example without limitation, be performed by contacting the tissue sample with an aqueous solution, with or without agitation, and with or without blending, for a delipidizing period of time, followed by separation and recovery of delipidized tissue from the aqueous solution. The aqueous solution comprises water and may, but does not have to, further contain one or more of: an organic solvent (e.g., a paraffin, an aromatic hydrocarbon, a cyclic hydrocarbon, a chlorinated or fluorinated hydrocarbon, an alcohol, an ether, a ketone, an organic acid, an aldehyde, an ester, and combinations thereof), an organic acid (e.g., a mineral acid), an organic base (e.g., a mineral base), an organic salt (e.g., a mineral salt).

At least one step of (D) milling the tissue sample is performed before, during, after, or a combination thereof, one or more other steps of the method. Milling the tissue sample (D) is, of course, performed to reduce the tissue sample to sizes smaller than the previously described step of (B) reducing the size of the tissue sample (e.g., by cutting the tissue into strips or smaller pieces), but is also performed according to parameters which retain, minimize or avoid destruction of, or even enhance, at least a portion of the native fibrous structure of the tissue sample. The parameters of each (D) milling step include a milling period of time and a speed, typically stated in revolutions per minute (rpm).

For example, without limitation, a suitable and effective milling period of time may be from about 1 minute to about 30 minutes, including any one or more ranges or values therebetween and including the stated endpoints, such as about 1-15 minutes, or about 5-30 minutes, or about 5-20 minutes, or about 5-15 minutes, or about 10-30 minutes, or about 10-20 minutes, or about 15-20 minutes, or about 10-15 minutes.

For example, but without limitation, a suitable and effective milling speed may be from about 1,000 rpm to about 10,000 rpm, including any one or more ranges or values therebetween and including the stated endpoints, such as about 1,000-8,000 rpm, such as about 1,000-6,000 rpm, such as about 1,000-5,000 rpm, or about 2,000-8,000 rpm, or about 2,000-5,000 rpm, or about 2,000-4,000 rpm, or about 1,500-3,500 rpm, or about 2,500-4,000 rpm, or about 3,000-4,500 rpm.

In some embodiments, a milling step (D) may include two or more substeps or phases, each of which comprises different combinations of milling periods of time and milling speeds, generally within the broadest ranges stated above. In one exemplary embodiment, without limitation, the step of (D) milling the tissue sample may comprise performing a first phase in which the tissue is milled for about 10-30 seconds at about 1000-3000 rpm, followed by a second phase in which the tissue is further milled for about 45 seconds to about 3 minutes at about 3000-4500 rpm.

It will be recognized that there are many possible embodiments of the method in which one or more milling steps (D) may suitably and successfully be performed at various possible points during the method, to produce a fibrous tissue derived material. For example, without limitation, relative to the step of delipidizing (C) the tissue sample, milling (D) may be performed: one or more times before delipidizing (C), one or more times during delipidizing (C), one or more times before and during delipidizing (C), one or more times after delipidizing (C), one or more times during and after delipidizing (C), one or more times before and after delipidizing (C), or one or more times before, during, and after delipidizing (C).

Furthermore, without limitation and relative to the step of decellularizing (F), milling (D) may be performed: one or more times before decellularizing (F), one or more times during decellularizing (F), one or more times before and during decellularizing (F), one or more times after decellularizing (F), one or more times during and after decellularizing (F), one or more times before and after decellularizing (F), or one or more times before, during, and after decellularizing (F).

Furthermore, the step of milling (D) the tissue sample may, for example without limitation, suitably and successfully be performed: one or more times before both delipidizing (C) and decellularizing (F), one or more times in between delipidizing (C) and decellularizing (F), one or more times after both delipidizing (C) and decellularizing (F), or one or more times before delipidizing (C) and one or more times before and during decellularizing (F).

Additionally, without limitation and relative to the step of disinfecting (G), milling (D) may be performed: one or more times before disinfecting (G), one or more times during disinfecting (G), one or more times before and during disinfecting (G), one or more times after disinfecting (G), one or more times during and after disinfecting (G), one or more times before, during, and after disinfecting (G).

Milling (D) may be performed: one or more times before both delipidizing (C) and disinfecting (G), one or more times in between delipidizing (C) and disinfecting (G), one or more times after both delipidizing (C) and disinfecting (G), one or more times before both delipidizing (C) and decellularizing (F), one or more times in between delipidizing (C) and decellularizing (F), one or more times after both delipidizing (C) and disinfecting (G). one or more times in between delipidizing (C) and disinfecting (G) one or more times after one or more of delipidizing (C), decellularizing (F), and disinfecting (G), or one or more times after all of delipidizing (C), decellularizing (F), and disinfecting (G).

In some embodiments, the step of (D) milling the tissue sample is performed by milling the tissue sample one or more times, before, during, or after the step of (F) decellularizing the tissue sample, or a combination of before, during, and after decellularizing. For example, without limitation, the step of (D) milling the tissue sample may be performed by (D) milling the tissue sample at least one time before the step of (F) decellularizing the tissue sample, as well as at least one time (D) after the step of (F) decellularizing the tissue sample. In some embodiments, no milling is performed prior to the step of (F) decellularizing the tissue sample, and the step of (D) milling the tissue sample is performed by milling the tissue sample at least one time (D) after the step of (F) decellularizing the tissue sample.

In some embodiments, the step of (D) milling the tissue sample is performed by milling the tissue sample concurrently with the step of (F) decellularizing the tissue sample, in which case the tissue will be combined or mixed with an aqueous solution comprising a decellularizing substance (as described in further detail below). Furthermore, in such embodiments, one or more additional steps of (D) milling the tissue sample may performed before, after, or both before and after the step of (F) decellularizing the tissue sample.

To retain or enhance the native fibrous structure of the tissue sample, and in accordance with the method described and contemplated herein, manipulation of one or more of milling parameters, including but not limited to time, speed, iterations, and raising the pH of the tissue sample, may be performed to control the degree to which the native fibrous structure of the tissue sample is retained, enhanced, or both, in the fibrous tissue sample material product. Controlling or limiting one or more of the time, speed, and iterations during each (D) milling step performed affects and controls the degree to which the fibrous quality of the tissue sample is retained, enhanced, or both.

Additionally, it has been found that a more fibrous product may be produced by raising the pH of the tissue sample, from acidic (i.e., less than about 6.5 pH) to neutral (i.e., from about 7.2 to 7.7 pH), either before, during, or both before and during, (D) milling of the tissue sample. As previously stated, raising pH (i.e., reducing the acidity) of the tissue sample before or during one or more milling steps may be accomplished by contacting the tissue sample with a buffered aqueous solution in any of several ways. For example, without limitation, the tissue sample may be contacted with a buffered aqueous solution, to raise the pH during milling by: contacting or mixing a buffered aqueous solution, instead of water, with the tissue sample in one or more existing milling steps, or by adding one or more additional milling steps wherein a buffered aqueous solution is mixed or contacting with the tissue sample, or both.

Another technique for raising the pH of a tissue sample to be milled is performing a pre-mill soaking step by mixing, or otherwise contacting and combining, the tissue sample with a buffered aqueous solution and then pausing or waiting a soaking period of time, prior to commencing a milling step (D). Such a pre-mill soaking step may be performed once, prior to a milling step (D), or in connection with multiple milling steps (D), such as once prior to each of two or more milling steps (D).

Additionally, any of the one or more steps of (D) milling the tissue samples may, but is not required to, be performed using a device or technique which reduces or allows control of damage to the native fibrous structure of the tissue sample. Such techniques and devices include, for example without limitation, blending or milling using a cutting type of blending or milling device having one or more knifes or blades (e.g., but without limitation, a GRINDOMIX GM 300 knife mill, commercially available from Retsch USA at Verder Scientific, Inc., located in Newtown, Pennsylvania, U.S.A.). Typically, blending and milling are performed on a tissue sample which has been combined or mixed with an aqueous solution or solvent.

Buffered aqueous solutions suitable for use in a pre-mill soaking step include water, or one or more buffered solutions such as, without limitation, phosphate-buffered saline (PBS), Dulbecco's phosphate-buffered saline (DPBS) (e.g., concentration 0.25×-10×), 2-(N-morpholino) ethanesulfonic acid (MES), tris(hydroxymethyl)aminomethane (Tris), TRIZMA® buffer (commercially available from ATA Bioquest, located in Pleasanton, California, U.S.A.), 4-(2-hydroxyethyl)-1-piperazineethanesulfonic acid (HEPES), (3-(N-morpholino) propanesulfonic acid (MOPS), 2-Hydroxy-3-morpholinopropanesulfonic acid (MOPSO), Bis(2-hydroxyethyl)amino-tris(hydroxymethyl) methane (Bis-Tris), 2,2′-[(2-amino-2-oxoethyl) azanediyl]diacetic acid (ADA), N-(2-acetamido)-2-aminoethanesulfonic acid (ACES), 2,2′-(Piperazine-1,4-diyl)di(ethane-1-sulfonic acid) (PIPES), 2-(bis(2-hydroxyethyl)amino) ethane sulfonic acid (BES), 2-{[1,3-Dihydroxy-2-(hydroxymethyl) propan-2-yl]amino}ethane-1-sulfonic acid (TES), 3-(N,N-Bis [2-hydroxyethyl]amino)-2-hydroxypropanesulfonic acid (DIPSO), 4-(N-Morpholino) butanesulfonic acid (MOBS), 3-[1,3-dihydroxy-2-(hydroxymethyl) propan-2-yl]amino]-2-hydroxypropane-1-sulfonic acid (TAPSO), N-(Hydroxyethyl) piperazine-N′-2-hydroxypropanesulfonic acid (HEPPSO), Piperazine-1,4-bis(2-hydroxypropanesulfonic acid)dihydrate (POPSO), Triethanolamine (TEA), 4-(2-Hydroxyethyl)-1-piperazinepropanesulfonic acid (EPPS), N-(Tri(hydroxymethyl)methyl)glycine (Tricine), Glycyl-glycine (Gly-Gly), Diethylolglycine (Bicine), N-(2-Hydroxyethyl) piperazine-N′-(4-butanesulfonic acid (HEPBS), N-Tris(hydroxymethyl)methyl-3-aminopropanesulfonic acid (TAPS), 2-Amino-2-methyl-1,3-propanediol (AMPD), N-tris(Hydroxymethyl)methyl-4-aminobutanesulfonic acid (TABS), N-(1,1-Dimethyl-2-hydroxyethyl)-3-amino-2-hydroxypropanesulfonic acid (AMPSO), N-Cyclohexyl-2-aminoethanesulfonic acid 3-(Cyclohexylamino)-2-hydroxy-1-(CHES), propanesulfonic acid (CAPSO), 2-methyl-2-amino-1-propanol (AMP), 3-(Cyclohexylamino) propane-1-sulfonic acid (CAPS), 4-(Cyclohexylamino)-1-butanesulfonic acid (CABS), and combinations thereof.

It should also be noted that the pH of the tissue sample to be subjected to one or more milling steps to produce the fibrous tissue derive material may also or alternatively, be manipulated during the pre-mill soaking step by addition of acidic or basic solutions, alone or in combination with one or more buffers. In one embodiment, for example without limitation, pH of the tissue sample may be increased or raised by adding a base, such as sodium hydroxide (NaOH) to the solution used in the pre-mill soaking step. Additionally, manipulating the pH may be accomplished during one or more milling steps by addition acidic or basic solutions to the solutions combined with the tissue sample for the milling steps.

Without wishing to be limited by theory, it is believed that either milling the tissue sample in one or more buffered solutions, or performing a step of pre-mill soaking with the tissue sample in combined or mixed with one or more buffered solutions, increases the pH of the tissue sample, which reduces acidity. Increased pH and reduced acidity of the tissue sample, in turn, reduces swelling of the tissue sample during and after milling, which ultimately retains more of the native fibrous structure and increases cohesiveness of the resulting fibrous tissue derived material.

Additionally, at any point during performance of the method described and contemplated herein, if desired, the step of (E) removing aqueous solution or solvent from the tissue sample may be performed before the next step of the method is performed. Such a step of (E) removing aqueous solution or solvent from the tissue sample may, for example without imitation, be performed by sieving, gravitational settling following by decanting, centrifuging, and combinations thereof. For example, without limitation, in an embodiment of the method in which a step of (D) milling the tissue sample in an alcohol solvent is performed (i.e., the tissue sample would be combined or mixed with a quantity of an aqueous alcohol solution, followed by milling), it may be advantageous to perform a step of (E) removing at least some of the alcohol solvent from the milled tissue-alcohol mixture prior to performing another step of (D) milling the tissue sample after combining or mixing with additional alcohol or another aqueous solvent.

The step of (F) decellularizing the tissue sample removes at least a portion of the endogenous cells and cellular material from a tissue sample, which reduces or minimizes the immunogenicity of the tissue sample, thereby, providing a highly biocompatible tissue derived material. The technique or method of decellularizing the tissue sample is not particularly limited and may include any technique known now or in the future to persons of ordinary skill in the relevant art. Techniques which preserve the native (natural) fibrous structure of the tissue sample are preferred. For example, without limitation, decellularizing may be performed by contacting the tissue sample with one or more of the following decellularizing substances: highly acidic solutions, highly basic solutions, hypertonic solutions, bypotonic solutions, alcohols, and detergents, for a decellularizing period of time. Other suitable decellularizing techniques include physical methods including, without limitation, applying pressure, cyclic freeze-thaw. Combinations of any one or more decellularizing techniques are possible and contemplated.

As will be determinable by persons of ordinary skill in the relevant art, based on general knowledge and informed by the present disclosure, the decellularizing techniques and substances are selected and used in concentrations and with other treatment parameters (e.g., with or without agitation, mixing speeds and time periods, contact time periods, etc.), and based on the nature of the technique and substance, to control damage or destruction of the collagenous matrix of the tissue being processed to produce fibrous tissue derived material having the desired degree of fibrousness.

In some embodiments, the step of (F) decellularizing the tissue sample may be performed by contacting, whether concurrently or sequentially, the tissue sample with one or more decellularizing substances, such as one or more decellularizing agents, or a biocompatible aqueous solution which is, or contains, one or more decellularizing agents. In some embodiments, the step of (F) decellularizing the tissue sample may comprise sequentially, or concurrently, contacting the tissue sample with a solution comprising one or more decellularizing substances.

In some embodiments, the step of (F) decellularizing the tissue sample comprises contacting the tissue sample with a solution comprising a hypotonic solution, such as without limitation sodium chloride (e.g., 1M NaCl) for a decellularizing period of time of from about 6 hours to about 72 hours, including any one or more ranges or values therebetween and including the stated endpoints, such as for example, about 10-48 hours, or about 6-36 hours, or about 12-36 hours, or about 12-24 hours, or about 6-24 hours, or about 10-40 hours. In some embodiments, the step of (F) decellularizing the tissue sample comprises contacting the tissue sample with a solution comprising a detergent, such as without limitation TRITON® X100 (commercially available from Millipore Sigma, located at Burlington, Massachusetts, U.S.A.), or a similarly soluble and chemically active nonionic surfactant, for a decellularizing period of time of from about 12 hours to about 72 hours, such as for example, about 12-36 hours, or about 24-72 hours, or about 24-48 hours.

In some embodiments, the step of (F) decellularizing the tissue sample comprises first contacting the tissue sample with a NaCl solution for a first decellularizing period of time of from about 12 to about 24 hours, followed by contacting the tissue sample with an aqueous solution of TRITON X100 for a second decellularizing period of time of from about 24 to about 48 hours, or from about 30 hours to about 40 hours. The NaCl solution may, for example without limitation be from about 0.1M to about 10M or any one or more ranges or values therebetween and including the stated endpoints, such as from about 0.5M to about 5M, or even from about 0.5M to about 1.5M. In some embodiments, the aforesaid step of first contacting the tissue sample with a IM NaCl solution also comprises milling the tissue sample and NaCl mixture.

Optionally and typically, but not necessarily, performed after decellularizing (F), the method for producing the fibrous tissue derived material may further comprise the step of (G) disinfecting the tissue sample, by techniques known now or in the future to persons of ordinary skill in the relevant art, for decreasing the amount, or removing substantially all, of the microbes, bacteria, and other infectious substances from the tissue. Such techniques may be, without limitation, chemical, mechanical, exposure to radiation, etc., or any combination thereof.

In some embodiments, the step (G) of disinfecting the tissue sample comprises contacting the soft tissue with a disinfecting solution for a disinfecting period of time, with or without agitation (e.g., stirring, shaking, blending, etc.). The disinfecting solution may, without limitation, include one or more of the following disinfecting agents: antibiotics, alcohols, a glycol, a peroxy compound (e.g., peracetic acid, PAA), chlorine dioxide, a detergent or surfactant. an ethylene diamine salt (e.g., ethylene diamine tetraacetic acid (EDTA)).

In some embodiments, during the step of (G) disinfecting, the tissue sample is in contact with the disinfecting solution for a disinfecting period of time of from about 1 hour to about 4 hours. In some embodiments, for example without limitation, the tissue sample is disinfected (G) by contact with an aqueous solution comprising PAA for a disinfecting period of time such as from about 30 minutes to about 4 hours, any one or more ranges or values therebetween and including the stated endpoints, including without limitation from about 1 hour to about 3 hours, or from about 2 to about 4 hours, or from about 2 hours to about 3 hours.

After the disinfecting step (G), the disinfected tissue sample may be washed or rinsed with water or another aqueous solution to remove the disinfecting solution. For example, without limitation, the disinfected tissue sample may be washed or rinsed with a buffer solution to remove the disinfecting solution.

The step of (H) combining the tissue sample with one or more solvents or solutions may comprise, but is not limited to, one or more of the following techniques: contacting, adding, pouring, mixing, stirring, agitating, shaking, blending, milling, and combinations thereof. The step of (H) combining the tissue sample with one or more solvents or solutions may be performed for any of several reasons and purposes such as, without limitation, to hydrate the tissue sample or, depending on the solvent or solution, to alter the pH of the tissue sample. The step of (H) combining the tissue sample with one or more solvents or solutions may be performed as an integral but not specifically stated part of another method step, such as adding a decellularizing solution to the tissue sample as part of the decellularizing step (F), which may or may not further include mixing, stirring, agitating, or a combination thereof, the tissue-solution mixture.

Furthermore, the step of (H) combining the tissue sample with one or more solvents or solutions may, in some embodiments, be performed as part of rinsing or soaking the tissue sample with the one or more solvents or solutions. Rinsing and soaking the tissue sample may performed with or without stirring or other agitation.

For example, without limitation, rinsing may be performed for removing and separating a previously applied solvent or solution, such as a decellularization solution or disinfecting solution, from the tissue sample. Accordingly, rinsing may, therefore, comprise a combination of sequentially (H) combining the tissue with a solvent or solution, and removing (I) the solvent or solution, along with for example decellularizing agents or disinfecting agents, by one or more techniques selected from, but not limited to, filtering, decanting, sieving, pouring, centrifuging and combinations thereof.

When the step of (H) combining the tissue sample with one or more solvents or solutions is performed for soaking the tissue sample, the tissue sample and solvent or solution will be allowed to remain in contact for a soaking period of time, such as from about 6 hours to about 72 hours, any one or more ranges or values therebetween and including the stated endpoints. For example, without limitation, a suitable and effective soaking time may be about 6-60 hours, or about 6-48 hours, or about 12-48 hours, or about 12-36 hours, or about 12-24 hours, or about 24-48 hours. The selected soaking time will, of course, vary depending on the type of solvent or solution, the desired effect or purpose, and the point at which soaking is performed (e.g., what method steps are performed before and after the combining step (H) which comprises soaking).

It should be understood that, in some embodiments, the step of (D) milling the tissue sample is performed by milling the tissue sample one or more times, after the step of (F) decellularizing the tissue sample, but before performing the step of (G) disinfecting the tissue sample. In some embodiments, the step of (D) milling the tissue sample is performed by milling the tissue sample at least one time after (D2) decellularizing (F) the tissue sample, and before disinfecting (G) the tissue sample. Regardless of any prior milling steps being performed, one or more milling steps (D) may be performed after both decellularizing (F) and disinfecting (G) the tissue sample. In some embodiments, one or more milling steps (D) are performed only after both decellularizing (F) and disinfecting (G) the tissue sample.

In some embodiments of the method for making the fibrous tissue derived material, after decellularizing (F), optionally disinfecting (G), and one or more milling steps, the tissue sample may be further processed, such as by (H) combining the tissue sample with one or more solvents, followed by (J) dehydrating (e.g., by lyophilizing) the tissue sample to produce fibrous tissue derived material. Such further processed fibrous tissue derived material may be in a three-dimensional shape which is incidental to a container in which it was dehydrated, or which is a desired or predetermined shape formed by placing the material and solvent mixture in a mold prior to dehydrating. In some embodiments, after (H) combining the fibrous tissue derived material with one or more solvents or solutions, if desired or advantageous, at least a portion of the one or more solvents just combined (H) with the material may be separated (I) prior to performing the step of (J) dehydrating the fibrous tissue derived material.

The resulting fibrous tissue derived material has one or more properties including, without limitation, porous, compressible, cohesive, wickable, absorbent, and when at least partially (or fully) hydrated, moldable, putty-like, shapable, cohesive, retains its shape, flowable, and injectable.

Methods for making a composition comprising a fibrous tissue derived material are also described and contemplated herein. Method for making such compositions comprise the steps of:
(1) obtaining a fibrous tissue derived material;
(2) optionally, adding one or more additional materials;
(3) optionally, adding one or more solvents or solutions to the fibrous tissue derived material and one or more additional materials; and
(4) forming a shaped body of the fibrous tissue derived material, with or without using a mold.

Such compositions are compressible and have shape retention. After rehydration of the lyophilized fibrous tissue derived material, the resulting composition is putty-like and moldable into a shape or other conformity to a void (wound, defect, etc.) being treated. The composition possesses sufficient structure, compressive strength, and shape retention, after implanting in the void, whereby the composition takes on the shape of the void. Furthermore, the implanted composition retains compressive strength to maintain its post-implantation shape for a period of time long enough for cells to infiltrate and incorporate, and maintain any restored volume provided by tissue matrix production as mentioned above.

In some embodiments, the method of making a composition comprising a fibrous tissue derived material further comprises: performing the step of (2) adding the one or more additional materials before, after, or both before and after, the step of (3) forming a shaped body of the fibrous tissue derived material.

In some embodiments, the method for making a composition comprising the fibrous tissue derived material comprises combining one or more additional materials with the fibrous tissue derived material. Combining one or more additional materials with the fibrous tissue derived material may, for example without limitation, include mixing, contacting, adsorbing, infusing, attaching, or otherwise combining the fibrous tissue derived material with the one or more additional materials.

Suitable additional materials are each biocompatible and may, without limitation, include one or more of: biocompatible non-tissue material, including but not limited to polymers (natural or synthetic), ceramics, metals, nature-derived or animal-derived biomaterials. Compositions comprising the fibrous tissue derived materials may also contain additional materials such as, without limitation, endogenous beneficial substances such as growth factors, extracellular matrix components, nutrients, biologically active molecules, vitamins, or integrins which facilitate various tissue healing and remodeling mechanisms including, without limitation, extracellular matrix production and deposition, cell infiltration and proliferation, pathogen barrier and reduction, and angiogenesis. Furthermore, the compositions may comprise a fibrous tissue derived material coated with, infused with, or otherwise including exogenous substances or materials, including without limitation, cells, growth factors, extracellular matrix components, nutrients, integrins, anti-microbial agents, anti-infective agents, bacteriostatic agents, or other substances such as, but not limited to, those which promote cell migration, attachment, proliferation, growth and activity.

The fibrous tissue derived material and compositions comprising them may be placed or implanted at a wound site, in contact with host tissue, as a bioresorbable porous component of a reduced pressure therapy wound treatment system. Some embodiments of the fibrous tissue derived material and compositions comprising them intended for use in reduced pressure therapy wound treatment may, for example without limitation, have porosity of about 50-1000 um, any one or more ranges or values therebetween and including the stated endpoints, such as about 100-600 um, or about 150-400 um. Additionally, fibrous tissue derived materials having more elongated fibers are expected to help prevent the fibrous tissue derived material body or compositions comprising same from being sucked up into the NPWT vacuum system and, thereby, remain in the wound after placement.

Furthermore. the fibrous tissue derived material and compositions comprising it are useful as dressings, grafts, scaffolds, etc., applied to wound sites and will facilitate and enhance wound healing and remodeling, even in the absence of reduced pressure therapy. Such uses generally include placement (i.e., implantation) of a fibrous tissue derived material and compositions comprising it, in contact or proximity, with a wound site of a subject wherein the matrix is resorbable and has a porosity which allows fluid flow through the material or composition. Additionally, the fibrous tissue derived material and compositions comprising it, as described and contemplated herein, are useful for treatment of a subject to restore, enhance, add to, or replace tissues in any area of the subject's body that requires support, restoration, regeneration, enhancement, or replacement.

It should be understood that the causes or origins of conditions and tissue sites to be treated using the fibrous tissue derived materials are not particularly limited, but rather may be any of several causes and origins including, but not limited to trauma, disease, surgery, other prior treatments and procedures, natural or congenital anomalies, defects, deficiencies, and cosmetically or aesthetically undesirable or disfavored conditions. Accordingly, the fibrous tissue derived materials are useful and provide improved properties and results for several categories of treatments including, but not limited to aesthetic or cosmetic treatments, reconstructive treatments, and wound treatments.

For example, fibrous tissue derived material and compositions comprising it, as described and contemplated herein, are useful for wound healing, treatment, and management, such as providing a matrix to cover or pack (fill) wounds and other defects with substantial depth to rebuild a healing bed, either alone, or in combination with other therapies and treatments (e.g., skin graft, application of placental materials, dressings). Wounds of substantial depth, tunneling wounds, chronic healing-resistant wounds, ulcers, traumatic wounds caused by accidents or even surgical procedures performed to treat other primary conditions (e.g., debridement of deep wounds or burns, tumor excision, cosmetic conditions, etc.), as well as other types of wounds, may be effectively treated using the fibrous tissue derived materials, with or without other materials, devices and substances.

Furthermore, fibrous tissue derived material and compositions comprising it are expected to be useful, even without additional treatment therapies, to treat irregular shaped wounds, for application to or implanting in difficult to access wound sites and tunneled wounds. As previously mentioned, in all of the aforesaid uses, treatments, and applications, fibrous tissue derived material and compositions comprising it, as described and contemplated herein, will provide a scaffold for cellular invasion and capillary growth.

It is noted that the fibrous tissue derived material and compositions comprising it will be useful and effective in the aforesaid treatments and applications even without crosslinking or freeze-drying (lyophilizing). Lyophilizing or other dehydration methods will produce fibrous tissue derived material, as described and contemplated herein, which are easy to ship and store at room or ambient temperatures, for extended periods of time (e.g., more than a week, or even up to several months).

The fibrous tissue derived material and compositions comprising it may also be used in plastic and soft tissue reconstruction where soft tissue defects exist, thickness and volumes may need to be restored, or where bulking with a biocompatible and remodeling natural biomaterial is needed. Such treatments and conditions including volume restoration will sometimes involve a breast, a hand, post-flap deficiencies, along with others. Furthermore, the causes and origins of defects and deficits in thickness and volume of soft tissues that are treatable with the fibrous tissue derived materials are not limited, but sometimes include trauma (accident, surgical, etc.), disease, congenital or other natural conditions, among others. Specifically, soft tissue deficits and other conditions caused by fibrosis, radiation fibrosis, resection procedures, excision of tumors (e.g., lumpectomy), removal of foreign bodies, excision of abnormal, excessive, or otherwise undesirable soft tissue, biopsy sites, flap procedures, autologous donor sites and more, are effectively and successfully treated by administration of one or more fibrous tissue derived materials and compositions comprising them, as disclosed and contemplated herein.

Additional contemplated and potential uses for the fibrous tissue derived material and compositions comprising it include providing large volumes of graft material useful as a filler or a bulking agent for body contouring and reconstruction, such as in the breast, buttocks, abdomen, buttocks, legs, face, or for tumor removal deficits (including small deficits for example from lumpectomy procedures, as well as large deficits for example from mastectomy procedures). The fibrous tissue derived materials and compositions comprising them are beneficial and effective for use in volume augmentation and volume enhancement treatments. In some embodiments, the fibrous tissue derived material may be decellularized and, therefore, lack immunogenicity and be highly biocompatible. Additionally, the fibrous tissue derived material and compositions comprising it are capable of providing needed bulk, support, barrier function, and padding, for treatment subjects having experienced prior tissue loss and/or destruction, regardless of the cause.

The fibrous tissue derived material and compositions comprising it are also expected to be useful and effective as scaffolds for regenerative medicine, especially when combined with cells, exosomes, growth factors, or other biological compound to further the regeneration of the defect tissue, as well as for regenerating or engineering skin. As previously suggested, additional materials such as, without limitation, antibiotics, antimicrobial agents, antifungal agents, antiviral agents, and combinations thereof, may be added, mixed or otherwise combined with the fibrous tissue derived material to produce compositions which provide added therapeutic properties especially useful when treating wounds and damaged or diseased tissue at high risk of infection.

It will be understood that the embodiments of the present invention described hereinabove are merely exemplary and that a person skilled in the art may make variations and modifications without departing from the spirit and scope of the invention. All such variations and modifications are intended to be included within the scope of the present invention.

### EXAMPLES

Several exemplary embodiments of methods for producing fibrous tissue derived material, as well as the material produced thereby, are described below. Reference will be made to the several figures of this disclosure to facilitate description and understanding of the invention described and contemplated herein.

### Example 1—Mill at End in Water

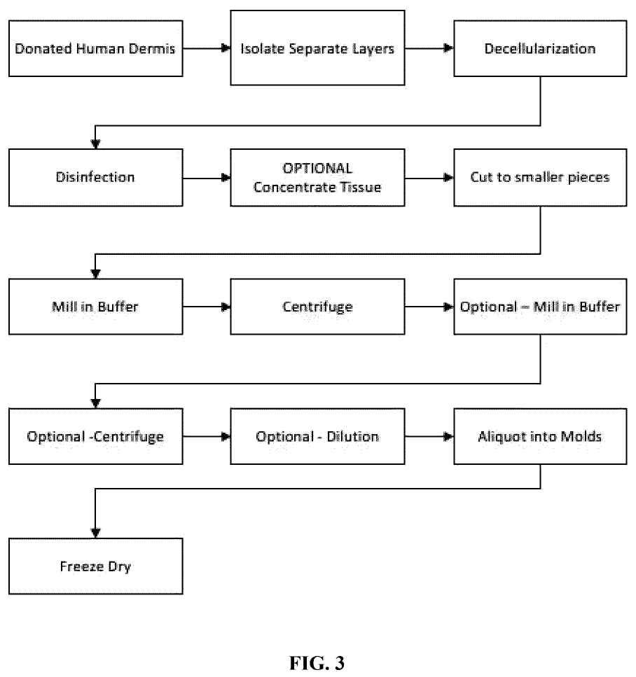

An exemplary embodiment of the method for producing fibrous tissue derived material, in which milling was performed at the end of the method (i.e., after decellularizing and disinfecting) was performed, generally as shown in the flowchart of FIG. 3. As detailed below, a modified version of the method shown in FIG. 3 was performed in which the milling iterations were performed in water (rather than buffer as indicated in FIG. 3) and after milling in buffer, followed by centrifuging, two additional milling steps (in water) were performed after centrifuging (suggested by “optional-mill” step).
(A) Sheet(s) of reticular dermis were isolated from a full thickness human dermis tissue sample donations.
(F1) The sheet(s) were decellularized via a 12-24 hour soak in 1M NaCl solution
(F2) The sheet(s) were decellularized via a 24-48 hour soak in 0.1% Triton solution, followed by a sequence of water rinses to remove residual chemical solution.
(G) The sheet(s) were then disinfected via a 2-4 hour soak in a PAA solution.
(E) Perform sequential water rinses to remove residual chemical solution.
(B) The sheet(s) were next cut to approximately 2×2 cm square reticular dermis pieces.
(D) The pieces were combined with water at a ratio of 125 g of tissue to 300 mL of water in the container of the GM300 knife mill. The pieces were (D1) milled via a sequence of a first milling at 2500 rpm for 20 seconds, followed by (D2) a second milling at 4000 rpm for 2 minutes.
(E) The resulting fibrous tissue mixture was then centrifuged at 4600 rpm for 5 minutes to remove excess water.
(D) The concentrated tissue was added to the container of the GM 300 knife mill with 300 mL of water. A second iteration of the milling sequence (D3), (D4), followed by centrifugation at the same parameters stated above, were performed.

The concentrated tissue was added to the GM 300 container with 300 mL of water. A final third iteration of the milling sequence (D5), (D6) at the same parameters stated above was performed.

A 1 g sample of the resulting fibrous tissue mixture was taken and used for SEM imaging.

FIG. 1 provides the SEM image taken of the 1 g sample, in which the fibrous structure of the fibrous tissue derived material, that was retained from the dermal tissue sample, can be seen.

### Example 2—Milling Before, During and after Decellularizing, and During Disinfecting

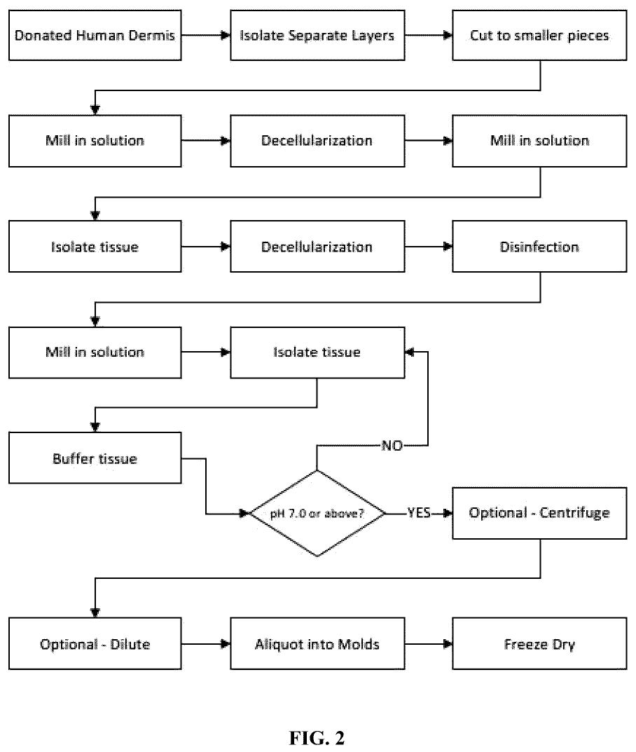

An exemplary embodiment of the method for producing fibrous tissue derived material, in which milling is performed throughout the method, is shown in FIG. 2 and described below.
(A) Obtain donated dermis, isolate layers of donated dermis tissue which includes reticular dermis.
(B) Cut isolated dermis to 2×2 cm pieces.
(D), (F1) Decellularize tissue pieces by blending (milling) tissue in 1M NaCl (e.g., concentration of about 0.1-0.2 g/mL, tissue to NaCl), first (D1) for 10-30 seconds(s) at 1500-3000 rpm, then (D2) for a total of 1-3 minutes at 3000-4000 rpm with 15-30 s intervals.
(D), (F2) Let blended tissue soak in NaCl for 6-48 hours with agitation on an orbital shaker; then (D3) blend (mill) again first (D3) for 10-30 seconds(s) at 1500-3000 rpm, then (D2) for a total of 1-3 minutes at 3000-4000 rpm with 15-30 s intervals.
(I) Centrifuge the blended tissue to form concentrated tissue pellet.
(F3) Decellularize by soaking tissue pellet in about 0.5-1 L 0.1% Triton for 12-48 hours, with agitation on an orbital shaker.
(E) Soak in 1 L water with agitation on an orbital shaker for 5-30 min, optionally, 6-10 times, with sieving in between water soaks.
(G1) Disinfect the decellularized milled tissue sample by soaking in PAA solution e.g., concentration of about 0.1-0.3 g/mL, tissue to PAA) for 1-4 hours, with agitation on an orbital shaker. The disinfected milled tissue at end of disinfecting PAA soaking phase is very fibrous.
(D), (G2) Blend (mill) soaked tissue in PAA, with small amount of 1-10×DPBS (buffered aqueous solution) added, where milling is performed first (D5) for 10-30 seconds(s) at 1500-3000 rpm, then (D6) for a total of 1-3 minutes (min) at 3000-4000 rpm with 15-30 s intervals. [Since the tissue is very fibrous before this blending/milling in PAA, instead the 3000-4000 rpm blend, more of the native fibrous structure of the tissue could be retained in the final fibrous tissue derived material by performing blending/milling at 1500-3000 rpm.]
(I) Centrifuge the disinfected milled tissue to form tissue pellet.
(E) Soak disinfected tissue pellet in about 0.5-1 L of 1×-10×DPBS for 5-15 min with agitation on an orbital shaker.

Verify whether the tissue soaked in DPBS has pH 7.0 or greater:
if not, repeat immediately preceding centrifuge and soak and,
if yes, go on to optional centrifuging.
(I) Optionally, centrifuge the disinfected milled tissue to form tissue pellet.
(H) Optionally, dilute the tissue by addition of water to produce a diluted water-tissue mixture having concentration of tissue to water of from about 80:20 to about 40:60, or any range or concentration therebetween, such as without limitation from about 70:30 to about 50:50.
(J) Pour the diluted water-tissue mixture in molds and freeze dry (lyophilize).

Lyophilizing is performed to make the resulting fibrous tissue derived material easily stored at temperatures above zero, with an extended shelf life, and capable of rehydration by the clinician at the time of use. After concentration, and before or after aliquoting, a known volume of solution may be added to the fibrous tissue derived material. This may be done to give the material and a mass, body, or graft formed therefrom, more consistent volume and the capability of being formed into a useful shape such as a cone, or puck, or cube, or even sheet. The addition of water may also allow increasing the porosity of the material and a mass, body, or graft formed therefrom, for faster rehydration (e.g., more pore space allows a rehydration solution to penetrate the material or graft made thereof more easily than a collapsed concentrated dry mass of fibers).

### Example 3—Milling after Decellularizing and Disinfecting

Generally, performing the method using milling steps according to parameters including, but not limited to, one or more of lower speeds (rpm), less milling time, and fewer milling steps or phases, is expected to produce a more fibrous tissue derived material which retains or includes enhanced fibrous structure from the tissue samples.

An exemplary embodiment of the method for producing fibrous tissue derived material, in which milling is performed after decellularizing and disinfecting, as well as in the presence of a buffered aqueous solution, is shown in FIG. 3 and described below.
(A) Obtain donated dermis, isolate layers of donated dermis tissue which includes reticular dermis.
(F1) Decellularize tissue sheets by soaking in 1M NaCl for 12-48 hours with agitation in an orbital shaker at 90 rpm (e.g., concentration of about 600-1,200 cm²/L, tissue to NaCl).
(F2) Decellularize by soaking tissue sheet in 0.1% Triton for 24-48 hours, with agitation in an orbital shaker at 90 rpm (e.g., concentration of about 600-1,200 cm²/L, tissue to Triton).
(H1) Soak in 1 L water, with agitation on an orbital shaker for 5-30 min, optionally, for example, 8 times, with sieving in between water soaks.
(G) Disinfect the decellularized tissue sheet by soaking in PAA solution (e.g., concentration of about 187.5-375 cm²/L, tissue to PAA), for 2-4 hours, with agitation on an orbital shaker.
(H2) Soak in 8 L water, with agitation on an orbital shaker, for 5-45 min, optionally, 8 times, with sieving in between water soaks.
(E) Optionally, removing water to concentrate the decellularized disinfected tissue.
(B) Cut decellularized, disinfected dermis tissue sheets into approximately 2×2 cm pieces
(D1) Blend/mill tissue pieces in 0.75-10×DPBS (e.g., concentration of about 0.1-0.3 g/mL, tissue to DPBS, for 0.5-2 min at 1000-4000 rpm.
(I1) Optionally, centrifuge the blended/milled tissue to form concentrated tissue pellet.
(D2) Optionally, blend/mill tissue in 0.75-10×DPBS (e.g., concentration of about 0.1-0.3 g/mL, tissue to DPBS), for 0.5-2 min at 1000-4000 rpm.
(I2) Optionally, centrifuge the blended/milled tissue to form concentrated tissue pellet.
(H) Optionally, dilute concentrated tissue by addition of water to produce a diluted water-tissue mixture having concentration of tissue to water of from about 80:20 to about 40:60, or any range or concentration therebetween, such as without limitation from about 70:30 to about 50:50.
(D3) Optionally, blend/mill diluted water-tissue mixture in water again for 10-30 sec at 1000-3000 rpm.
(J) Pour diluted water-tissue mixture in molds and freeze dry.

### Example 4A—Milling in Alcohol and Buffer, after Decellularizing and Disinfecting

Milling after both decellularizing and disinfecting the tissue is expected to result in more fibrous tissue derived material. Generally, adding milling steps with tissue sample in alcohol (e.g., propanol) prior to milling in water improves the consistency and concentration of the final fibrous tissue derived material prior to dilution.

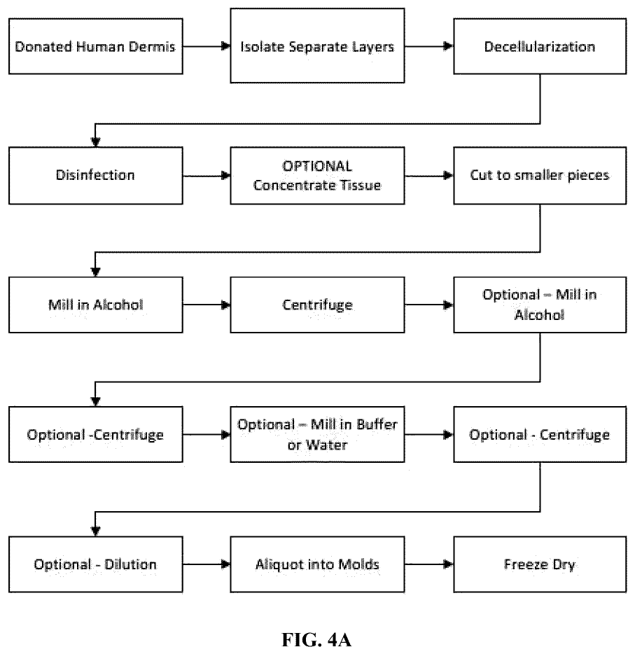

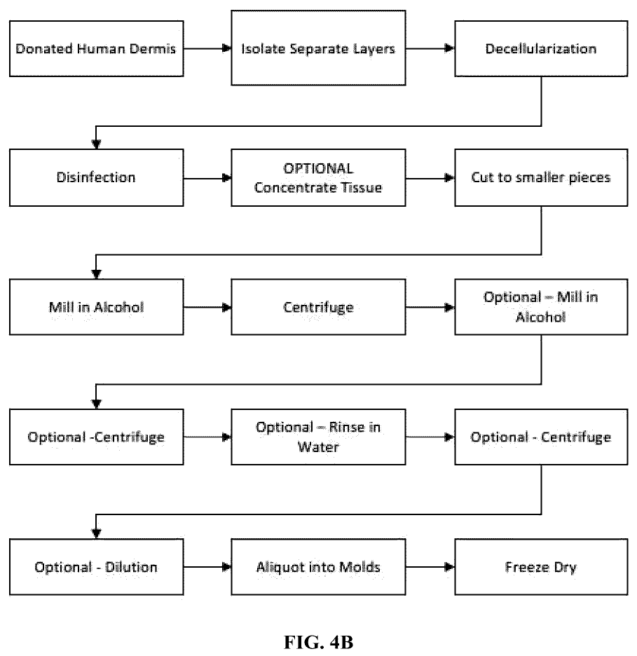

An exemplary embodiment of the method for producing fibrous tissue derived material, in which milling is performed in alcohol (e.g., propanol) after decellularizing and disinfecting, followed by milling in the presence of a buffered aqueous solution, is shown in FIG. 4A and described below.
(A) Obtain donated dermis, isolate layers of donated dermis tissue which includes reticular dermis.
(F1) Decellularize tissue sheets by soaking in 1M NaCl for 12-24 hours with agitation in an orbital shaker at 90 rpm (e.g., concentration of about 600-1,200 cm²/L, tissue to NaCl).
(F2) Decellularize by soaking tissue sheets in 0.1% Triton for 24-48 hours, with agitation in an orbital shaker at 90 rpm (e.g., concentration of about 600-1,200 cm²/L, tissue to Triton).
(H1) Soak in 1 L water, with agitation in an orbital shaker at 90 rpm, for 5-30 min, optionally, 8 times, with sieving in between water soaks.
(G) Disinfect the decellularized tissue sheets by soaking in PAA solution (e.g., concentration of about 187.5-375 cm²/L, tissue to PAA), for 2-4 hours, with agitation in an orbital shaker at 90 rpm.
(H2) Soak in 8 L water, with agitation in an orbital shaker at 90 rpm, for 5-15 min, optionally, 8 times, with sieving in between water soaks.
(E) Optionally, removing water to concentrate the decellularized disinfected tissue.
(B) Cut decellularized, disinfected dermis tissue sheets into 2×2 cm pieces
(D) Blend/mill tissue pieces in 50% 1-propanol (e.g., concentration of about 0.1-0.3 g/mL, tissue to 1-propanol), first (D1) for about 20 sec at 1000-3000 rpm, then (D2) for 1 min at 3000-4000 rpm.
(I1) Centrifuge the blended/milled tissue to form concentrated tissue pellet.
(D) Optionally, blend/mill tissue pieces in 50% 1-propanol (e.g., a concentration of about 0.1-0.3 g/mL, tissue to 1-propanol), first (D3) for 20 sec at 1000-3000 rpm, then (D4) for 1 min at 3000-4000 rpm.
(I2) Optionally, centrifuge the blended/milled tissue to form concentrated tissue pellet.
(D5) Blend/mill tissue pieces in 0.75-10×DPBS (e.g., a concentration of about 0.1-0.3 g/mL, tissue to DPBS), for 0.5-2 min at 1000-4000 rpm.
(I1) Optionally, centrifuge the blended/milled tissue to form concentrated tissue pellet.
(D6) Optionally, blend/mill tissue in 0.75-10×DPBS (e.g., a concentration of about 0.1-0.3 g/mL, tissue to DPBS), for 0.5-2 min at 1000-4000 rpm.
(I2) Optionally, centrifuge the blended/milled tissue to form concentrated tissue pellet.
(H) Optionally, dilute concentrated tissue by addition of water to produce a diluted water-tissue mixture having concentration of tissue to water of from about 80:20 to about 40:60, or any range or concentration therebetween, such as without limitation from about 70:30 to about 50:50.
(D3) Optionally, blend/mill diluted water-tissue mixture in water again for 10-30 sec at 1000-3000 rpm.
(J) Pour diluted water-tissue mixture in molds and freeze dry.

### Example 4B—Milling in Alcohol, Soaking in Water, after Decellularizing and Disinfecting

Another exemplary embodiment of the method for producing fibrous tissue derived material, in which milling is performed in alcohol (e.g., propanol) after decellularizing and disinfecting, followed by soaking in water instead of further milling, is shown in FIG. 4B and described below.

After milling in propanol, the milled tissue is fibrous and, therefore, it may be advantageous to eliminate subsequent milling in water (as described above in Example 4A), and even to replace those subsequent milling steps with soaking steps in water for removing propanol from the tissue sample.
(A) Obtain donated dermis, isolate layers of donated dermis tissue which includes reticular dermis.
(F1) Decellularize tissue sheets by soaking in 1M NaCl for 12-24 hours with agitation in an orbital shaker at 90 rpm (e.g., concentration of about 600-1,200 cm²/L, tissue to NaCl).
(F2) Decellularize by soaking tissue sheets in 0.1% Triton for 24-48 hours, with agitation in an orbital shaker at 90 rpm (e.g., concentration of about 600-1,200 cm²/L, tissue to Triton).
(H1) Soak in 1 L water, with agitation in an orbital shaker at 90 rpm, for 5-30 min, optionally, 8 times, with sieving in between water soaks.
(G) Disinfect the decellularized tissue sheets by soaking in PAA solution (e.g., concentration of about 600-1,200 cm²/L, tissue to PAA), for 2-4 hours, with agitation in an orbital shaker at 90 rpm.
(H2) Soak in 8 L water, with agitation in an orbital shaker at 90 rpm, for 5-15 min, optionally, 8 times, with sieving in between water soaks.
(E) Optionally, removing water to concentrate the decellularized disinfected tissue.
(B) Cut decellularized, disinfected dermis tissue layers into 2×2 cm pieces.
(D) Blend/mill tissue pieces in 50% 1-propanol (e.g., a concentration of about 0.1-0.3 g/mL, tissue to 1-propanol), first (D1) for 20 sec at 2500 rpm, then (D2) for 1 min at 4000 rpm.
(I1) Centrifuge the blended/milled tissue to form concentrated tissue pellet.
(D) Optionally, blend/mill tissue pieces in 50% 1-propanol (e.g., a concentration of about 0.1-0.3 g/mL, tissue to 1-propanol), first (D3) for 20 sec at 2500 rpm, then (D4) for 1 min at 4000 rpm.
(I2) Centrifuge the blended/milled tissue to form concentrated tissue pellet.
(H3) Soak the blended/milled tissue in water (0.04-0.2 g/mL, tissue to water), for 5-30 min, with agitation on an orbital shaker (11) Optionally, centrifuge the soaked tissue to form concentrated tissue pellet.
(H4) Soak the tissue in water (0.04-0.2 g/mL, tissue to water), for 5-30 min, with agitation on an orbital shaker.
(I2) Optionally, centrifuge the soaked tissue to form concentrated tissue pellet.
(H5) Optionally, dilute concentrated tissue by addition of water to produce a diluted water-tissue mixture having concentration of tissue to water of from about 80:20 to about 40:60, or any range or concentration therebetween, such as without limitation from about 70:30 to about 50:50.
(D3) Optionally, blend/mill diluted water-tissue mixture in water again for 20 sec at 2500 rpm.
(J) Pour water-tissue mixture in molds and freeze dry.

### Example 5—Formation of Dehydrated Fibrous Tissue Sheets Following by Milling at End of Process

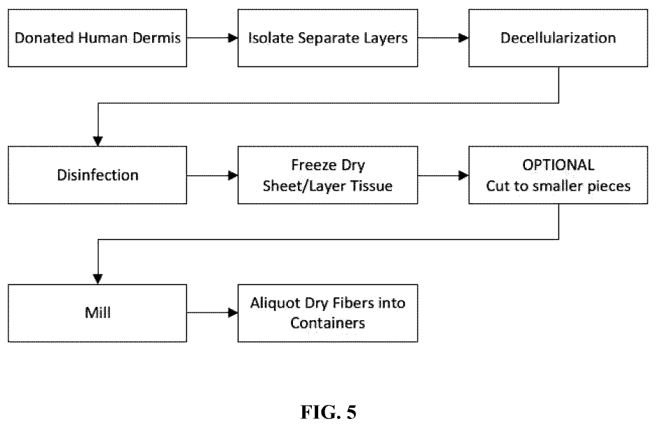

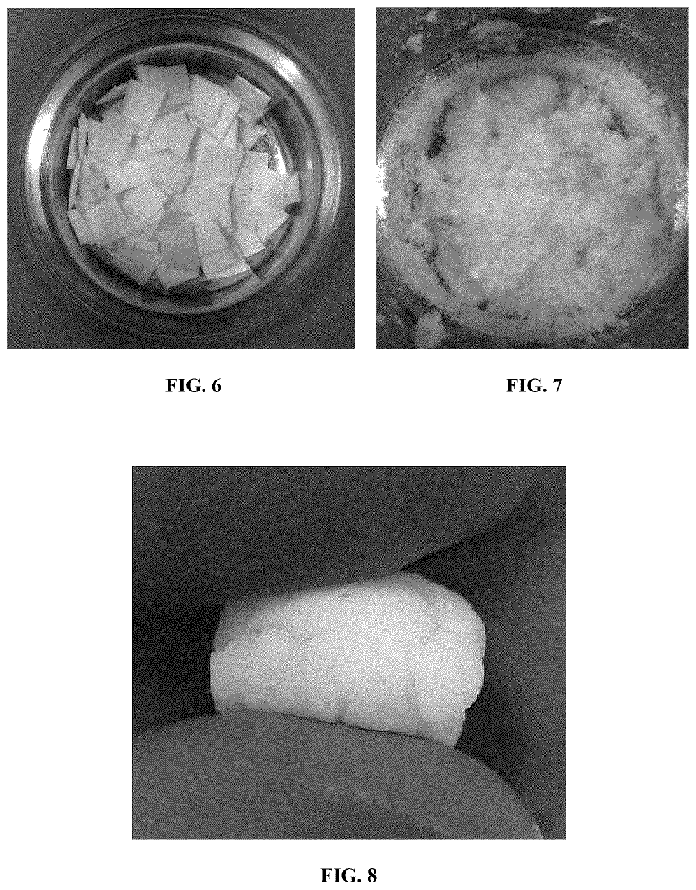

Yet another exemplary embodiment of the method for producing fibrous tissue derived material, in which dehydrated dermal tissue derived sheets are produced and then milled, is shown in FIG. 5 and described below.
(A) Donated dermis was obtained, layers of donated dermis tissue which includes reticular dermis were isolated.
(F1) Tissue sheets were decellularized by soaking in 1M NaCl for 12-24 hours with agitation in an orbital shaker at 65 rpm (i.e., concentration of 800 cm²/L of tissue to NaCl).
(F2) Tissue sheets were further decellularized by soaking in 0.1% Triton for 24-48 hours, with agitation in an orbital shaker at 65 rpm (i.e., concentration of 800 cm²/L of tissue to Triton).
(H1) Tissue sheets were rinsed in 1 L water, with agitation in an orbital shaker at 65 rpm, for 5-30 min, 8 times, with sieving in between water soaks.
(G) The decellularized tissue sheets were disinfected by soaking in PAA solution (i.e., 2380 cm² tissue/8 L PAA) for 2-4 hours, with agitation in an orbital shaker at 90 rpm.
(H2) The tissue sheets were rinsed in 8 L water, with agitation in an orbital shaker at 90 rpm, for 5-15 min, optionally, 8 times, with sieving in between water soaks.
(J) The sheets of decellularized, disinfected tissue were freeze dried.
(B) The freeze-dried decellularized, disinfected dermis tissue sheets were cut into approximately 1×1 cm pieces.
(D) The pieces of freeze-dried tissue were loaded into a mill (see FIG. 6) and milled at 10,000 rpm for 20 s pulses for a total of 8 pulses to produce a fibrous dermal tissue derived material (see FIG. 7).

Desired quantities (e.g., doses) of the resulting fibrous tissue derived material produced by the exemplary method of Example 5 may be placed into containers and sealed for storage and shipping.

With reference to FIGS. 6-8, the cut up pieces of the sheets of dehydrated dermis, which were produced in step (B) of Example 5, are shown in the mill in the photograph of FIG. 6, prior to milling. After performing milling step (D) of Example 5, the dehydrated fibrous dermal tissue derived material shown in the photograph of FIG. 7 was produced from the pieces shown in FIG. 7. Finally, the fibrous dermal tissue derived material shown in FIG. 7 was collected and manually shaped into a mass (or body) and then rehydrated by adding saline (i.e., 0.17 g fibrous material mixed with saline in a ratio of 30% w/w), to form the shapable putty shown in FIG. 8. The shapable putty is useful for administration during treatment for repairing and reconstructing diseased or otherwise damaged tissue.

The packaged fibrous dermis tissue derived material may be rehydrated by a clinician at the time of use to form a cohesive putty, or even placed in a wound dry (i.e., without rehydration) whereupon the fluid present at or which migrates to the wound would rehydrate the fibers, in situ, into a putty like material while the porous matrix of the fibrous dermal tissue derived material will promote and allow for cell infiltration and attachment.

### Example 6—Milling and Buffering Soaks after Decellularizing and Disinfecting

Another exemplary embodiment of the method for producing fibrous tissue derived material, in which milling was performed at the end of the method (i.e., after decellularizing and disinfecting) was performed, generally as shown in the flowchart of FIG. 3, and is described below. As detailed below, the method shown in FIG. 3 was modified such that, after decellularizing and disinfecting, one longer milling step, in a buffer, was performed, followed by 3 post-milling soaks in a buffer to raise the pH of the fibrous tissue derived material.
(A) Human dermis tissue samples were obtained and the reticular dermis layers were isolated.
(F1) The dermis tissue sheets were decellularized by soaking in 1M NaCl for 12-48 hours with agitation in an orbital shaker at 90 rpm (e.g., concentration of about 600-1,200 cm²/L, tissue to NaCl).
(F2) The dermis tissue sheets were further decellularized by soaking in 0.1% Triton for 24-48 hours, with agitation in an orbital shaker at 90 rpm (e.g., concentration of about 600-1,200 cm²/L, tissue to Triton).
(H1) The dermis tissue sheets were rinsed in 1 L water, with agitation on an orbital shaker for 5-30 min, 8 times, with sieving in between water soaks.
(G) The decellularized dermis tissue sheets were disinfected by soaking in PAA solution (e.g., concentration of about 187.5-375 cm²/L, tissue to PAA), for 2-4 hours, with agitation on an orbital shaker.
(H2) The disinfected dermis tissue sheets were rinsed in 8 L water, with agitation on an orbital shaker, for 5-45 min, 8 times.
(B) The decellularized, disinfected dermis tissue sheets were cut into approximately 2×2 cm pieces
(D1) The tissue pieces were blended/milled in 1×DPBS, at concentration of about 0.42 g/mL tissue to DPBS, for 3.3 minutes at 2500-4000 rpm.
(I1) The blended/milled tissue was centrifuged to form concentrated tissue pellet.
(E1) The blended/milled tissue was soaked in 500 mL of 1×DPBS for 5 min with agitation on an orbital shaker.
(I2) The blended/milled tissue was centrifuged to form concentrated tissue pellet.
(E2) The blended/milled tissue was soaked in 500 mL of 1×DPBS for 5 min with agitation on an orbital shaker.
(I3) The blended/milled tissue was centrifuged to form concentrated tissue pellet.
(E3) The blended/milled tissue was soaked in 500 mL of 1×DPBS for 5 min with agitation on an orbital shaker.
(I4) The blended/milled tissue was centrifuged to form concentrated tissue pellet.
(H) The concentrated tissue was diluted by addition of water to produce a diluted water-tissue mixture having concentration of tissue to water of 80:20.
(D2) The tissue mixture was blended/milled for 20 sec at 2500 rpm for mixing.
(J) the diluted water-tissue mixture was transferred to molds, photographed in the mold, and then freeze dried to produce dehydrated fibrous tissue derived material.

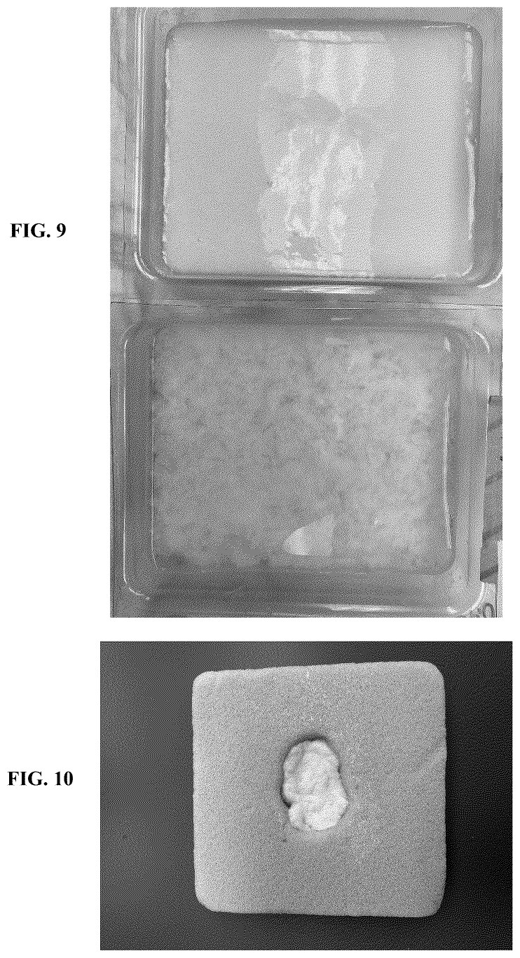

FIG. 9 is a photograph of a top mold/container tray containing comparative dermal tissue derived matrix produced by a method according to that disclosed in WIPO Publication No. WO2020227601 and outlined below, as well as a bottom mold/container containing the fibrous tissue derived material produced as described in Example 6. The more fibrous quality of the fibrous tissue derived material, i.e., comprised of thin, wispy, flexible, elongated, and at least partially intertwined strands of extracellular matrix. On the other hand, the dermal tissue derived matrix in the top mold/container shown in FIG. 9 is clearly less fibrous, generally having smaller fragments of extracellular matrix which forms a mass also useful for implanting in tissue defects as a graft, but less porous, cohesive, and capable or retaining its shape than the fibrous material in the top mold/container.

The comparative dermal tissue derived matrix shown in a mold/container in FIG. 9 was produced by a method disclosed in WO2020227601 which included decellularization and disinfection steps equivalent to those described throughout the foregoing examples, but also included several more aggressive (longer duration and/or higher spends) and more often repeated milling steps, as follows:
Donated dermis samples were obtained, and layers were separated to provide isolated dermal tissue sheets, including reticular dermis.
The tissue sheets were decellularized via soak in hypertonic solution for 12-24 hours with agitation.
The tissue sheets were further decellularized via soak in detergent solution for 24 hours with agitation.
The tissue sheets were rinsed via soak in water with agitation.
The decellularized tissue sheets were disinfected via soak in peracetic acid solution
for 2-4 hours with agitation.
The tissue sheets were rinsed via soak in water with agitation.
The decellularized, disinfected dermis tissue sheets were cut into approximately 2×2 cm pieces.
The pieces were blended/milled in 10%-100% 1-propanol (e.g., concentration of about 0.1-0.3 g/mL, tissue to solution) for approximately 3 min at 1000-4000 rpm.
The blended/milled tissue was centrifuged to form concentrated tissue pellet.
The tissue pellets were blended/milled in 300 mL of 10%-100% 1-propanol for approximately 3 min at 1000-4000 rpm.
The blended/milled tissue was centrifuged to form concentrated tissue pellet.
The tissue pellets were blended/milled in 300 ml of water for approximately 3 min at 1000-4000 rpm.
The blended/milled tissue was centrifuged to form concentrated tissue pellet.
The tissue pellets were blended/milled in 300 ml of water for approximately 3 min at 1000-4000 rpm.
The blended/milled tissue was centrifuged to form concentrated tissue pellet.
The concentrated tissue was diluted by addition of water to produce a diluted water
tissue mixture having concentration of tissue to water of from about 20:80.
The tissue mixture was blended/milled 10-30 sec at 1000-3000 rpm to mix homogenously.
The diluted water-tissue mixture was poured into molds/containers.

The photograph provided in FIG. 9 was taken after the diluted water-tissue mixture was formed and poured into the mold/container. The photograph of the comparative (prior art) dermal tissue derived matrix and the presently disclosed fibrous tissue derived material was taken prior to dehydration (freeze drying) because in this form the differing fibrous qualities are more visually apparent. Dehydrating (freeze drying) both the comparative matrix and the fibrous material produce compositions which, when rehydrated, are reconstituted to forms having less and more fibrous qualities, respectively, that are equivalent to those shown in FIG. 9.

The increased porosity, cohesiveness, shape retention and compressibility, as compared to the prior art dermal tissue derived matrix, are demonstrated in FIG. 10 which is a photograph of the rehydrated fibrous tissue derived material shaped and inserted into (and filling) a simulated tissue defect, without any loose strands or material extending out of the defect.

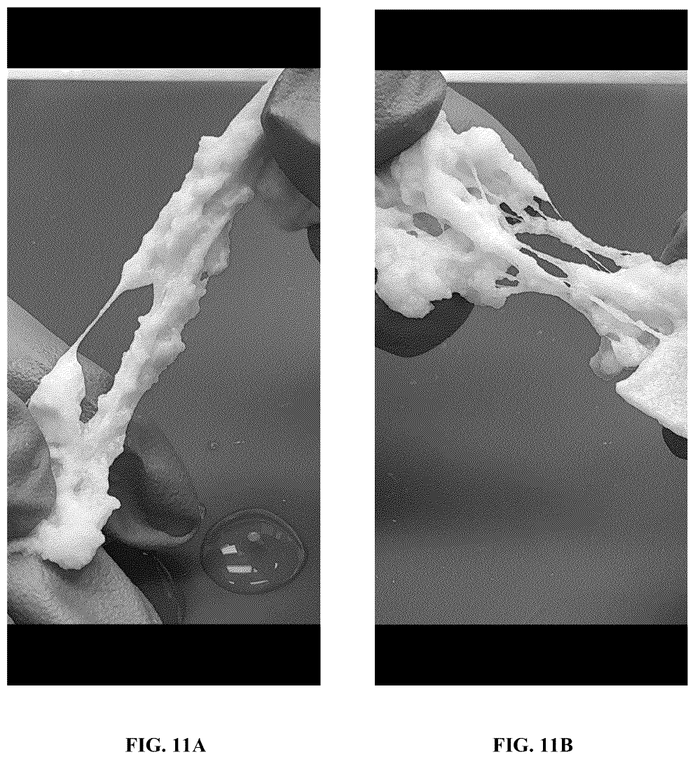

FIGS. 11A and 11B are photographs of the fibrous tissue derived material, produced by the method of this Example 6 and fully rehydrated with saline, being manually handled and manipulated. The fibrous quality and moldability and cohesive properties of the fibrous tissue derived material are clearly apparent in the photographs of FIGS. 11A and 11B.

We claim:

1. A fibrous tissue derived material having a fibrous structure formed by extracellular matrix strands derived from processing one or more tissue samples, each of which comprises one or more soft tissues or portions thereof.

2. The fibrous tissue derived material of claim 1, wherein the fibrous structure comprises thin, wispy, flexible, elongated, and at least partially intertwined strands of the extracellular matrix, wherein the fibrous structure has a porosity greater than porosities of the one or more soft tissues of the one or more tissue samples, and wherein the porosity of the fibrous structure allows for fluid flow therethrough and facilitates migration and infiltration by fluids, bioactive substances, cells, and other beneficial materials, after administration of the fibrous tissue derived material to a treatment site.

3. The fibrous tissue derived material of claim 1, wherein the one or more soft tissues or portions thereof, comprise one or more of dermis, adipose, fascia, muscle, and combinations thereof.

4. The fibrous tissue derived material of claim 3, wherein the one or more soft tissues or portions thereof consist essentially of: about 1-99% dermis tissue and about 1-99% fascia tissue, not to exceed a total of 100% and based on the total weight of the tissue derived material.

5. The fibrous tissue derived material of claim 1, wherein the fibrous tissue derived material is at least partially decellularized.

6. The fibrous tissue derived material of claim 1, wherein the fibrous tissue derived material is at least partially dehydrated.

7. The fibrous tissue derived material of claim 1, wherein the fibrous tissue derived material has one or more properties including: porous, compressible, cohesive, wickable, absorbent, moldable, shapable, cohesive, retains its shape, and combinations thereof.

8. The fibrous tissue derived material of claim 1, wherein the fibrous tissue derived material is at least partially hydrated or rehydrated and has one or more properties including: porous, compressible, cohesive, wickable, absorbent, putty-like, moldable, shapable, cohesive, retains its shape, flowable, injectable, and combinations thereof.

9. A composition comprising the fibrous tissue derived material of claim 1 and further comprising one or more biocompatible fluids.

10. The composition of claim 9, further having one or more properties including: porous, compressible, wickable, absorbent, putty-like, moldable, shapable, cohesive, retains its shape, flowable, injectable, and combinations thereof.

11. A composition comprising the fibrous tissue derived material of claim 1 and having a three-dimensional shape which is simple, complex, or a combination thereof, having increased cohesiveness, in the substantial absence of crosslinking, as compared to a less fibrous tissue derived materials, and having porosity which enables and facilitates cell infiltration after administration to a treatment site.

12. Method of treating a soft tissue condition comprising administration of the fibrous tissue derived material or a composition comprising same to a treatment site of a subject, wherein the fibrous tissue derived material supports and enhances soft tissue healing, remodeling, and reconstruction.

13. A method for producing a tissue derived material having a fibrous structure and comprising extracellular matrix derived from processing one or more tissue samples, wherein the method comprises obtaining the one or more tissue samples, each of which comprises one or more soft tissues or portions thereof, each soft tissue having a native fibrous structure, followed by the step of:
reducing the sample size of the one or more tissue samples one or more times, wherein at least one of the one or more times comprises performing one or more milling iterations using a milling device to produce the tissue derived material having a fibrous structure, wherein each of the one or more milling iterations is performed having milling parameters which retain, minimize or avoid destruction of, enhance, or a combination thereof, at least a portion of the native fibrous structure of one or more of the soft tissues in the one or more tissue samples; and
at least partially decellularizing the one or more tissue samples by performing one or more decellularizing steps, sequentially, concurrently, or a combination thereof, wherein each of the one or more decellularizing steps comprises chemical decellularizing, physical decellularizing, or a combination thereof, and is different or the same as other decellularizing steps.

14. The method of claim 13, wherein one or more milling iterations is performed before decellularizing, during decellularizing, before and during decellularizing, after decellularizing, during and after decellularizing, before and after decellularizing, or before, during, and after decellularizing.

15. The method of claim 13, further comprising increasing pH and thereby reducing acidity of the one or more tissue samples by either:
combining one or more buffered aqueous solutions with the one or more tissue samples to produce a buffer-tissue mixture and subjecting the buffer-tissue mixture to at least one milling iteration, or
performing at least one pre-mill soaking step by combining one or more buffered aqueous solutions with the one or more tissue samples to produce a buffer-tissue mixture and pausing a soaking period of time prior to subjecting the buffer-tissue mixture to at least one milling iteration, or
performing at least one post-mill soaking step by combining one or more buffered aqueous solutions with the one or more tissue samples to produce a buffer-tissue mixture and pausing a soaking period of time prior to performing further processing steps.

16. The method of claim 13, wherein each of the one or more milling iterations comprises either:
milling parameters which include a milling period of time and a milling speed, and wherein the milling period of time is different from or the same as that of other milling iterations and the milling speed is different from or the same as that of other milling iterations, or two or more milling phases, each of which comprises milling parameters which include a milling period of time and a milling speed, and wherein the milling period of time is different from or the same as that of other milling phases and the milling speed is different from or the same as that of other milling phases.

17. The method of claim 13, wherein at least one of the one or more milling iterations comprises:
combining the one or more tissue samples with an aqueous solution selected from water, a buffered aqueous solution, or an alcohol solution, or a combination thereof, prior to operating the device at the milling parameters; and
optionally removing at least a portion of the aqueous solution after completion of operating the device during each milling iteration,
wherein the aqueous solution combined with the one or more tissue samples during a milling iteration is the different from or the same as the aqueous solution using in each of the other one or more milling iterations.

18. The method of claim 17, wherein removing a least a portion of the aqueous solution after completion of operating the device is performed by sieving, gravitational settling followed by decanting, centrifuging, and combinations thereof.

19. The method of claim 13, wherein the device used during at least one of the one or more milling iterations has one or more knifes, blades, and linear, arcuate, or circular cutting edges, or a combination thereof.

20. The method of claim 13, further comprising forming a composition comprising the fibrous tissue derived material and having a three-dimensional shape which is simple, complex, or a combination thereof, wherein the composition has increased cohesiveness, in the substantial absence of crosslinking, as compared to a less fibrous tissue derived materials, and has porosity greater than porosities of the one or more soft tissues of the one or more tissue samples and which facilitates cell infiltration after administration to a treatment site.
